(19) United States
(12) Patent Application Publication
(10) Pub. No.: US 2012/0027799 A1
(43) Pub. Date: Feb. 2, 2012

(54) COMPOSITIONS AND METHODS FOR TREATING OR PREVENTING INFLAMMATORY BOWEL DISEASE AND COLON CANCER

(75) Inventors: Cynthia L. Sears, Baltimore, MD (US); Drew M. Pardoll, Brookeville, MD (US); Shaoguang Wu, Lutherville, MD (US); Franck Housseau, Ashburn, VA (US)

(73) Assignee: THE JOHNS HOPKINS UNIVERSITY, Baltimore, MD (US)

(21) Appl. No.: 13/262,449

(22) PCT Filed: Apr. 2, 2010
(86) PCT No.: PCT/US10/29767

§ 371 Date: Sep. 30, 2011

Related U.S. Application Data
(60) Provisional application No. 61/166,087, filed on Apr. 2, 2009.
(60) Provisional application No. 61/229,569, filed on Jul. 29, 2009.

(51) Int. Cl. A61K 39/07; G01N 33/50; A61P 37/04; G01N 33/53; C07K 16/12; C12Q 1/68; G01N 33/68

(52) U.S. Cl. 424/246.1; 435/6.12; 436/94; 436/86; 436/501; 530/389.5

(57) ABSTRACT

The invention provides compositions and methods for useful for the diagnosis of inflammatory bowel disease, ETBF-induced colitis, colonic hyperplasia and/or colon carcinogenesis in a subject in biological samples (e.g., stool, urine, blood, serum, tissue). The invention further provides compositions and methods for the treatment or prevention of colitis, colon cancer, or inflammatory bowel disease (e.g., Crohn's disease).

### CROSS-REFERENCE TO RELATED APPLICATION

This application claims the benefit of the following U.S. Provisional Application Nos. 61/166,087, filed Apr. 2, 2009, and 61/229,569, filed Jul. 29, 2009, the entire contents of which are incorporated herein by reference.

### STATEMENT OF RIGHTS TO INVENTIONS MADE UNDER FEDERALLY SPONSORED RESEARCH

This work was supported by the following grants from the National Institutes of Health, Grant Nos: RO1 DK45496 and RO1 DK080817. The government has certain rights in the invention.

### BACKGROUND OF THE INVENTION

Infection-associated inflammatory processes are known to enhance carcinogenesis in the affected organs. In humans, for example, chronic hepatitis (hepatitis B virus or hepatitis C virus) leads to liver cancer, and chronic Helicobacter pylori infection leads to gastric cancer in some individuals. Increased cancer incidence is likewise found in experimental mouse models of both infection-induced and noninfectious inflammation. The role of infectious and inflammatory processes in colon carcinogenesis is of considerable interest, as ˜1×10¹³ commensal bacteria colonize the colon, with inflammation resulting if colonic epithelial homeostasis is disrupted. Indeed, ulcerative colitis results in predictable development of colon cancer over time. Colorectal cancer is the third most common cancer diagnosed in both men and women in the United States, and the leading cause of cancer-related deaths in men and women. Therefore, improved methods of treating or preventing colon cancer, as well as underlying inflammatory conditions that contribute to carcinogenesis are urgently required.

### SUMMARY OF THE INVENTION

As described below, the present invention features compositions and methods for the treatment of colon cancer and/or inflammatory bowel disease (e.g., Crohn's disease, colitis).

In one aspect, the invention generally provides a method of diagnosing a subject (e.g., human) as having, or having a propensity to develop inflammatory bowel disease or colon carcinogenesis, the method involving detecting an enterotoxigenic B. fragilis (ETBF) nucleic acid molecule in a biological sample from a subject, where the presence of the ETBF nucleic acid molecule indicates that the subject has or has a propensity to develop inflammatory bowel disease or colon carcinogenesis.

In another aspect, the invention provides a method of diagnosing a subject (e.g., human) as having, or having a propensity to develop, inflammatory bowel disease or colon carcinogenesis, the method involving detecting an ETBF polypeptide in a subject sample, where the presence of ETBF polypeptide in the sample indicates that the subject has or has a propensity to develop inflammatory bowel disease or colon carcinogenesis.

In yet another aspect, the invention provides a method of diagnosing a subject (e.g., human) as having, or having a propensity to develop, inflammatory bowel disease or colon carcinogenesis, the method involving detecting an antibody against an ETBF polypeptide in a subject sample, where the presence of the antibody in the sample indicates that the subject has or has a propensity to develop inflammatory bowel disease or colon carcinogenesis. In one embodiment, the subject sample is blood, serum, or plasma. In another embodiment, the antibody is detected in an ELISA or other immunological assay. In another embodiment, the antibody specifically binds BFT-1, BFT-2, BFT-3 or a related isoform.

In yet another aspect, the invention provides a method of monitoring a subject (e.g., human) diagnosed as having inflammatory bowel disease or colon carcinogenesis, the method involving determining the level of an ETBF nucleic acid molecule or polypeptide in a subject sample, where an alteration in the level of expression relative to the level of expression in a reference indicates the severity of inflammatory bowel disease or colon carcinogenesis in the subject. In one embodiment, the nucleic acid molecule is bft-1, bft-2, bft-3, or a nucleic acid molecule that encodes a related isoform, and the polypeptide is BFT-1, BFT-2, BFT-3, or a related isoform. In another embodiment, the subject is being treated for inflammatory bowel disease or colon carcinogenesis. In another embodiment, the alteration is an increase, and the increase indicates an increased severity of inflammatory bowel disease or colon carcinogenesis. In another embodiment, the alteration is a decrease, and the decrease indicates a decreased severity of inflammatory bowel disease or colon carcinogenesis. In one embodiment, the reference is a control subject sample. In another embodiment, the reference is a subject sample obtained at an earlier time point.

In yet another aspect, the invention provides an ETBF antibody that specifically binds to an ETBF protein or fragment thereof. In one embodiment, the antibody specifically binds to BFT-1, BFT-2 and/or BFT-3 or another gene or polypeptide derived from ETBF.

In still another aspect, the invention provides a method for treating or preventing inflammatory bowel disease or colon carcinogenesis, the method involving administering to a subject an effective amount of an immunogenic composition containing a nucleic acid molecule (e.g., bft-1, bft-2, bft-3) encoding a ETBF protein or fragment thereof.

In yet another aspect, the invention provides a method for treating or preventing inflammatory bowel disease or colon carcinogenesis, the method involving administering to a subject (e.g., human) an effective amount of an immunogenic composition containing an ETBF protein (e.g., BFT-1, BFT-2, BFT-3, or a related isoform) or fragment thereof.

In still another aspect, the invention provides a method for treating or preventing inflammatory bowel disease or colon carcinogenesis, the method involving administering to a subject (e.g., human) an effective amount of a killed or attenuated ETBF cell (e.g., ETBF-1, -2, or -3). In one embodiment, the immunogenic composition is administered orally. In one embodiment, the effective amount is sufficient to induce an immune response in the subject.

In yet another aspect, the invention provides a method for producing an immune response against ETBF in a subject (e.g., human), the method involving administering to the subject an effective amount of an immunogenic composition containing an ETBF polypeptide, ETBF nucleic acid molecule, and/or killed or attenuated ETBF cell, thereby generating an immune response in the subject. In one embodiment, the immune response comprises production of neutralizing antibodies. In one embodiment, the nucleic acid molecule is present in an expression vector.

In yet another aspect, the invention provides a method for treating or preventing inflammatory bowel disease or colon carcinogenesis in a subject, the method involving administering to the subject an effective amount of a STAT3 inhibitor (e.g., mall compound or a STAT3 inhibitory nucleic acid molecule), thereby treating or preventing inflammatory bowel disease or colon carcinogenesis.

In yet another aspect, the invention provides a method for treating or preventing inflammatory bowel disease or colon carcinogenesis in a subject, the method involving administering to the subject an effective amount of an agent that reduces IL-17 biological activity, thereby treating or preventing inflammatory bowel disease or colon carcinogenesis. In one embodiment, the agent is an antibody that specifically binds IL-17 and blocks IL-17 binding to an IL-17 receptor or a soluble form of the IL-17R used as a decoy, or a drug that interferes with IL-17R signaling.

In yet another aspect, the invention provides a method for treating or preventing inflammatory bowel disease or colon carcinogenesis in a subject, the method involving administering to the subject an effective amount of an agent that reduces IL-23 binding to an IL-23 receptor, thereby treating or preventing inflammatory bowel disease or colon carcinogenesis. In one embodiment, the agent is an antibody that specifically blocks IL-23 binding to the IL-23 receptor or a soluble form of the IL-23R used as a decoy or a drug that interferes with IL-23R signaling.

In yet another aspect, the invention provides a method for treating or preventing inflammatory bowel disease or colon carcinogenesis in a subject, the method involving administering to the subject an effective amount of an agent that reduces the proliferation or survival of ETBF in a subject. In one embodiment, the agent is selected from the group consisting of metronizole, doxycycline, clindamycin, imipenem, meropenem, beta-lactam/beta-lactamase inhibitor combinations, cefotetan, tigecycline, moxifloxacin and derivatives of these classes of antibiotics.

In yet another aspect, the invention provides a method of identifying a compound that treats or prevents inflammatory bowel disease or colon cancer, the method involving contacting a cell that expresses an ETBF nucleic acid molecule or polypeptide with a candidate compound, and detecting a reduction in the level of expression of the nucleic acid molecule or polypeptide in the cell relative to the level of expression in a control cell, where a reduction in expression of the ETBF nucleic acid molecule or polypeptide identifies the candidate compound as a compound that treats or prevents inflammatory bowel disease or colon cancer.

In yet another aspect, the invention provides a method of identifying a compound that treats or prevents inflammatory bowel disease or colon cancer, the method involving contacting a cell that expresses an ETBF polypeptide with a candidate compound, and detecting a reduction in the biological activity of the ETBF polypeptide in the cell relative to the level in a control cell, where a reduction in ETBF biological activity identifies the candidate compound as a compound that treats or prevents inflammatory bowel disease or colon cancer.

In yet another aspect, the invention provides a method of identifying a compound that treats or prevents inflammatory bowel disease or colon cancer, the method involving contacting a colon-derived cell that expresses a STAT3, NFκB, MAPK or Wnt polypeptide with a candidate compound, and comparing the level of STAT3, NFκB, MAPK or Wnt expression or biological activity in the cell with the level of STAT3, NFκB, MAPK or Wnt expression or biological activity in a control cell, where a decrease in the expression or activity of the STAT3, NFκB, MAPK or Wnt polypeptide identifies the candidate compound as a compound that treats or prevents inflammatory bowel disease or colon cancer. In one embodiment, the cell is in vitro or in vivo. In another embodiment, the cell is a human cell. In another embodiment, the alteration is detected using an immunological assay, an enzymatic assay, or a radioimmunoassay.

In yet another aspect, the invention provides a diagnostic kit for the diagnosis of inflammatory bowel disease or colon carcinogenesis in a subject, the kit containing one or more primers for amplifying an ETBF nucleic acid molecule, or fragment thereof, and written instructions for use of the kit in any method delineated herein. In one embodiment, the nucleic acid molecule is bft-1, bft-2, bft-3 or a nucleic acid molecule that encodes a related isoform.

In yet another aspect, the invention provides a diagnostic kit for the diagnosis of inflammatory bowel disease or colon carcinogenesis in a subject, the kit containing an antibody that specifically binds an ETBF polypeptide, or fragment thereof, and written instructions for use of the kit in any method delineated herein. In one embodiment, the polypeptide is BFT-1, BFT-2, BFT-3 or a related isoform.

In yet another aspect, the invention provides a diagnostic kit for the diagnosis of inflammatory bowel disease or colon carcinogenesis in a subject, the kit containing an ETBF polypeptide bound to a substrate and directions for the use of the kit for the detection of an antibody that specifically binds to the ETBF polypeptide in a subject sample. In one embodiment, the ETBF polypeptide is BFT-1, BFT-2, BFT-3, or a related isoform. In another embodiment, the substrate is a plate for use in an ELISA assay.

In yet another aspect, the invention provides a kit for treating or preventing inflammatory bowel disease or colon carcinogenesis in a subject, the kit containing an ETBF polypeptide, ETBF nucleic acid molecule, or ETBF cell formulated as an immunogenic composition, and written instructions for use of the kit to induce an immune response in a subject.

In various embodiments of the above aspects or any other aspect of the invention delineated herein, the nucleic acid molecule is bft-1, bft-2, bft-3, or a nucleic acid molecule that encodes a related isoform. In other embodiments, the biological sample is a stool sample or blood sample. In other embodiments, the ETBF nucleic acid molecule is detected by PCR, qPCR, Northern blot, or probe hybridization. In other embodiments, the method detects an increase in the level of expression of the ETBF nucleic acid molecule relative to a reference. In other embodiments, the ETBF polypeptide is BFT-1, BFT-2, BFT-3, or a related isoform. In other embodiments, the method detects an increased level of the ETBF polypeptide relative to a reference. In other embodiments, the level of expression is determined in an immunological assay. In still other embodiments of the above aspects, the absence of an ETBF polypeptide or nucleic acid molecule indicates that the subject does not have inflammatory bowel disease or colon carcinogenesis or a propensity to develop such conditions. In still other embodiments of the above aspects, the method is used to diagnose a subject as having ETBF-induced colitis, inflammatory bowel disease, colonic hyperplasia or tumor formation. In still other embodiments of the above aspects, the method is used to determine the treatment regimen for a subject having inflammatory bowel disease or colon carcinogenesis. In still other embodiments of the above aspects, the method is used to monitor the condition of a subject being treated for inflammatory bowel disease or colon carcinogenesis. In still other embodiments of the above aspects, the method further comprises characterizing inflammation, hyperplasia and/or gastrointestinal intraepithelial neoplasia (GIN) foci in the subject. In other embodiments, the method further comprises characterizing phosphorylated Stat3 (pStat3) in intestinal mucosa of the subject. In other embodiments, an immunogenic composition of the invention is administered orally. In other embodiments, the method reduces colonic thickness, inflammation and/or visible colonic tumors.

The invention provides compositions and methods for prevention or treating ETBF-induced colitis, inflammatory bowel disease, colonic hyperplasia and tumor formation. Compositions and articles defined by the invention were isolated or otherwise manufactured in connection with the examples provided below. Other features and advantages of the invention will be apparent from the detailed description, and from the claims.

### DEFINITIONS

By “enterotoxigenic Bacteroides fragilis (ETBF)” is meant a pathogenic bacteria comprising a bft gene that is associated with ETBF-induced colitis, inflammatory bowel disease, colonic hyperplasia or tumor formation.

By “ETBF polypeptide” is meant any polypeptide encoded by the enterotoxigenic Bacteroides fragilis genome. In one embodiment, an ETBF polypeptide is a B. fragilis metalloproteinase toxin (BFT) having at least about 85% amino acid identity to NCBI Accession No. BAA77276.1, BAA77277.1, or BAA77275.1. BFTs are described, for example, by Kato et al., FEMS Microbiol. Lett. 182 (1), 171-176 (2000)

An exemplary BFT-1 amino acid sequence is provided below:

MKNVKLLLMLGTAALLAACSNEADSLTTSIDAPVTASIDLQSVSYTDLA
TQLNDVSDFGKMIILKDNGFNRQVHVSMDKRTKIQLDNENVRLFNGRDK
DSTSFILGDEFAVLRFYRNGESISYIAYKEAQMMNEIAEFYAAPFKKTR
AINEKEAFECIYDSRTRSAGKDIVSVKINIDKAKKILNLPECDYINDYI
KTPQVPHGITESQTRAVPSEPKTVYVICLRENGSTIYPNEVSAQMQDAA
NSVYAVHGLKRYVNFHFVLYTTEYSCPSGDAKEGLEGFTASLKSNPKAE
GYDDQIYFLIRWGTWDNKILGMSWFNSYNVNTASDFEASGMSTTQLMYP
GVMAHELGHILGAEHTDNSKDLMYATFTGYLSHLSEKNMDIIAKNLGWE
AADGD

An exemplary BFT-2 amino acid sequence is provided below:

MKNVKLLLMLGTAALLAACSNEADSLTTSIDTPVTASIDLQSVSYTDLA
TQLNDVSDFGKMIILKDNGFNRQVHVSMDKRTKIQLDNENVRLFNGRDK
DSTSFILGDEFAVLRFYRNGESISYIAYKEAQMMNEIAEFYAAPFKKTR
AINEKEAFECIYDSRTRSAGKDLVSVKINIDKAKKILNLPECDYINDYI
KTPQVPHGITESQTRAVPSEPKTVYVICLRESGSTVYPNEVSAQMQDAA
NSVYAVHGLKRFVNLHFVLYTTEYSCPSGNADEGLDGFTASLKANPKAE
GYDDQIYFLIRWGTWDNNILGISWLDSYNVNTASDFKASGMSTTQLMYP
GVMAHELGHILGARHADDPKDLMYSKYTGYLFHLSEENMYRIAKNLGWE
IADGD

An exemplary BFT-3 amino acid sequence is provided below:

MKNVKLLLMLGTAALLAACSNEADSLTTSIDAPVTASIDLQSVSYTDLA
TQLNDVSDFGKMIILKDNGFNRQVHVSMDKRTKIQLDNENVRLFNGRDK
DSTNFILGDEFAVLRFYRNGESISYIAYKEAQMMNEIAEFYAAPFKKTR
AINEKEAFECIYDSRTRSAGKYPVSVKINVDKAKKILNLPECDYINDYI
KTPQVPHGITESQTRAVPSEPKTVYVICLRENGSTVYPNEVSAQMQDAA
NSVYAVHGLKRYVNLHFVLYTTEYACPSGNADEGLDGFTASLKANPKAE
GYDDQIYFLIRWGTWDNNILGISWLNSYNVNTASDFKASGMSTTQLMYP
GVMAHELGHILGANHADDPKDLMYSKYTGYLFHLSEKNMDIIAKNLGWE
IADGD

By “ETBF nucleic acid molecule” is meant any nucleic acid molecule present in an enterotoxigenic Bacteroides fragilis genome. In one embodiment, an ETBF nucleic acid molecule is bft-1 (NCBI Accession No. AB026625), bft-2 (NCBI Accession No. AB 026626) or bft-3 (NCBI Accession No. AB 026624) or any other nucleic acid molecule encoding BFT-1, -2, or -3 or other related isoforms.

An exemplary bft-1 nucleic acid sequence is provided below:

| | |
|---|---|
| 1 | ttttaattaa actttaataa ttttaagata tattatggag tgctattcgg ttaaaatttg |
| 61 | cagtaggaat gcattatgag cagacaggca tcggtttgct gggcgtatat ggtgtttgga |
| 121 | tggcaagatt gggagattag cttagtaaaa tgtgcattgg gatcagggaa aatgtggaat |
| 181 | cacatcgtgc atcagtggaa atgggatgag tatcataaaa cgctgctctt tttccgttcc |
| 241 | atcttcagaa ctaatattga tatgtgtact attaatatgt tacatataat aatgaattgt |
| 301 | tgcaacaatt ataaaaccaa tgtttatttt aaattttaac aaaatgaaga atgtaaagtt |
| 361 | acttttaatg ctaggaaccg cggcattatt agctgcatgt tctaatgaag ctgattctct |
| 421 | aacaacatct attgatgctc cagttacagc ttccattgac ttacaatcag taagttatac |
| 481 | tgatttagcg acacaactta acgatgtatc ggactttggc aaaatgatta ttctaaaaga |
| 541 | caatggtttc aaccgtcagg tacatgtttc tatggataag cgtactaaaa tacagctgga |
| 601 | taatgagaat gtccgtctgt tcaacggcag ggacaaggat tctaccagct ttatactggg |
| 661 | agatgagttc gcagtattac gtttttatcg caatggcgaa tccatcagct acatcgcata |
| 721 | caaggaagcg caaatgatga atgagattgc cgaattttat gctgcaccat ttaaaaagac |
| 781 | acgtgcaata aacgagaagg aggcttttga atgcatttat gattcaagga caagaagtgc |
| 841 | tggaaaggat attgtttcag taaaaatcaa tattgacaag gcaaaaaaaa tattgaatct |
| 901 | tcctgaatgc gattatataa atgattacat aaaaacgcct caagtacctc atggaataac |
| 961 | tgaaagtcag acacgtgcag taccttctga acctaaaacg gtatatgtca tttgtctgag |
| 1021 | agagaatgga agtactatct atcctaatga agttagtgcc cagatgcagg atgcggcgaa |
| 1081 | ctcggtttat gcagttcatg gactgaaaag atatgtcaat ttccactttg tactgtatac |
| 1141 | tactgaatac agttgtccaa gtggcgacgc caaagaggga ctggaaggct ttactgcttc |
| 1201 | actaaaaagt aatccaaaag cagaaggtta tgacgatcaa atttattttt taatacgctg |
| 1261 | gggtacttgg gataataaaa tcttagggat gtcctggttc aattcttata atgtgaatac |
| 1321 | ggcttcggat tttgaagcca gtgggatgtc tacaacccag ctgatgtatc ccggggtgat |
| 1381 | ggcacacgaa ctaggtcata tactgggcgc tgagcatacg gataattcaa aagatttgat |
| 1441 | gtatgctaca tttactggat acttatccca tttgtccgag aaaaatatgg atataatcgc |
| 1501 | taaaaatctc ggttgggaag ctgcagatgg cgattagata aaaaag |

An exemplary bft-2 nucleic acid sequence is provided below:

| | |
|---|---|
| 1 | ttttaattaa actttgatga ttttaaaaca tgttatgaag tgctgttcgg ttaaaatttg |
| 61 | cagtaggaat gcattatgag cagacaggca tcggttgctg ggcgtatatg gtgtttggat |
| 121 | ggcaatgatt gggagattag cttagtaaaa tgtgcattgg gatcagggaa aatgtggaat |
| 181 | cacatcgtgc atcagtggaa atgggatgag tatcataaaa cggtgctctt ttttccgttc |
| 241 | catcttcaga actaatattg atatgtgtac tattaatatg ttacatataa taatgaattg |
| 301 | ttgcaacaat tataaaacca atgtttattt taaattttaa caaaatgaag aatgtaaagt |
| 361 | tacttttaat gctaggaacc gcggcattat tagctgcatg ttctaatgaa gctgattctc |
| 421 | taacaacatc tattgatact ccagttacag cttccattga cttacaatca gtaagttata |
| 481 | ctgatttagc gacacaactt aacgatgtat cggactttgg caaaatgatt attctaaaag |
| 541 | acaatggttt caaccgtcag gtacatgttt ctatggataa gcgtactaaa atacagctgg |
| 601 | ataatgagaa tgtccgtctg ttcaacggca gggacaagga ttctaccagc tttatactgg |
| 661 | gagatgagtt cgcagtatta cgtttttatc gcaatggcga atccatcagc tacatcgcat |
| 721 | acaaggaagc gcaaatgatg aatgagattg ccgaatttta tgctgcacca tttaaaaaga |
| 781 | cacgtgcaat aaatgagaag gaggcttttg aatgcattta tgattcaagg acaagaagtg |
| 841 | ctggaaagga tcttgtttca gtaaaaatca atattgacaa agccaagaaa atattgaatc |
| 901 | ttcctgaatg cgattatata aatgattaca taaaaacgcc tcaagtacct catggaataa |
| 961 | ctgaaagtca gacacgtgca gtaccttctg aacctaaaac ggtatatgtc atttgtctga |
| 1021 | gagagagtgg aagtactgtt tatcctaatg aggttagtgc ccagatgcag gatgcggcga |
| 1081 | actcggttta tgcagttcat ggactgaaaa gatttgtcaa tctccacttt gtactttata |
| 1141 | ctactgaata tagttgcccg agcggcaatg ccgatgaagg gctggatggc tttactgctt |
| 1201 | cactaaaagc taatccgaaa gcagaaggtt atgacgacca aatttatttt ttaatacgct |
| 1261 | ggggtacttg ggataacaac atcttaggca tatcttggct tgattcttac aatgtgaata |
| 1321 | cggcttcgga ttttaaagcc agtgggatgt ctacaaccca gctgatgtat cccggggtga |
| 1381 | tggcacacga actagggcat atattgggtg ctaggcatgc ggatgatcca aaagatttga |
| 1441 | tgtattctaa atatacggga tatttattcc atttgtccga ggagaacatg tatagaatcg |
| 1501 | ctaaaaatct cggatgggaa atagcagatg gcgattagat aaccaa |

An exemplary bft-3 nucleic acid sequence is provided below:

| | |
|---|---|
| 1 | ttttaattaa acttcggtaa ttttaaaata tattatggag tgctgttcgg ttaaaatttg |
| 61 | cagtaggaat gcattatgag cagacaggca tcggttgctg ggcgtatatg gtgtttggat |
| 121 | ggcaatgatt gggagattag cttagtaaaa tgtgcattgg gatcagggaa aatgtggaat |
| 181 | cacatcgtgc atcagtggaa atgggatgag tgtcataaaa cggtgctctt ttttccgttc |
| 241 | catcttcaga actaatattg atatgtatac tattaatatg ttacatataa taatgaattg |
| 301 | ttgcaacaat tataaaacca atgtttattt taaattttaa caaaatgaag aatgtaaagt |
| 361 | tacttttaat gctaggaacc gcggcattat tagctgcatg ttctaatgaa gctgattctc |
| 421 | taacaacatc tattgatgct ccagttacag cttccattga cttacaatca gtaagttata |
| 481 | ctgatttagc gacacaactt aacgatgtat cggactttgg caaaatgatt attctaaaag |
| 541 | acaatggttt caaccgtcag gtacatgttt ctatggataa gcgtactaaa atacagctgg |
| 601 | ataatgagaa tgtccgtctg tttaacggca gggacaagga ttctaccaac tttatactgg |
| 661 | gagatgagtt cgcagtatta cgtttttatc gcaatggcga atccatcagc tacatcgcat |
| 721 | acaaggaagc gcaaatgatg aatgagattg ccgaatttta tgctgcacca tttaaaaaga |
| 781 | cacgtgcaat aaacgagaag gaggcttttg aatgcattta tgattcaagg acaagaagtg |
| 841 | ctggaaagta tcctgtttca gtaaaaatca atgttgacaa agccaagaaa atattgaatc |
| 901 | ttcctgaatg cgattatata aatgattaca taaaaacgcc tcaggtacct catggaataa |
| 961 | ctgaaagtca gacacgtgca gtaccttctg aacctaaaac ggtatatgtc atttgtctga |
| 1021 | gagagaatgg aagtactgtt tatcctaacg aagttagtgc ccagatgcag gatgcggcga |
| 1081 | actcggttta tgcagttcat ggactgaaaa gatatgtcaa tctccacttt gtactttata |
| 1141 | ctactgaata tgcttgtccg agcggcaatg ccgatgaagg gctggatggc tttactgcct |
| 1201 | cattaaaagc taatccgaaa gcagaaggtt atgacgatca aatttatttt ttgatacgct |
| 1261 | ggggaacttg ggacaacaac attttgggca tatcttggct caattcttat aatgttaata |
| 1321 | cggcttcgga ttttaaagcc agtgggatgt ctacaaccca gctgatgtat cctggggtta |
| 1381 | tggcacacga actaggtcat atattgggtg ctaaccatgc ggatgatcca aaagatttga |
| 1441 | tgtattctaa atatacggga tatttattcc atttgtccga gaagaatatg gatataattg |
| 1501 | ctaaaaatct cggatgggaa atagcagatg gcgattagat aaaaata |

By “colon carcinogenesis” is meant colon cancer or a pathologic change in the colon that contributes to or precedes tumor formation. Such pathological changes include, but are not limited to colonic hyperplasia, dysplasia, and any other condition related to increased cellular proliferation.

By “STAT3 inhibitor” is meant any agent that reduces STAT3 biological activity.

By “STAT3 biological activity” is meant STAT3 transcriptional activity or any other STAT3 activity that contributes to carcinogenesis. STAT3 is a transcription factor that is either required for transformation, enhances transformation, or blocks apoptosis.

By “agent” is meant any small molecule chemical compound, antibody, nucleic acid molecule, or polypeptide, or fragments thereof.

By “ameliorate” is meant decrease, suppress, attenuate, diminish, arrest, or stabilize the development or progression of a disease.

By “alteration” is meant a change (increase or decrease) in the expression levels or activity of a gene or polypeptide as detected by standard art known methods such as those described herein. As used herein, an alteration includes a 10% change in expression levels, preferably a 25% change, more preferably a 40% change, and most preferably a 50% or greater change in expression levels.”

By “analog” is meant a molecule that is not identical, but has analogous functional or structural features. For example, a polypeptide analog retains the biological activity of a corresponding naturally-occurring polypeptide, while having certain biochemical modifications that enhance the analog's function relative to a naturally occurring polypeptide. Such biochemical modifications could increase the analog's protease resistance, membrane permeability, or half-life, without altering, for example, ligand binding. An analog may include an unnatural amino acid.

In this disclosure, “comprises,” “comprising,” “containing” and “having” and the like can have the meaning ascribed to them in U.S. patent law and can mean “includes,” “including,” and the like; “consisting essentially of” or “consists essentially” likewise has the meaning ascribed in U.S. patent law and the term is open-ended, allowing for the presence of more than that which is recited so long as basic or novel characteristics of that which is recited is not changed by the presence of more than that which is recited, but excludes prior art embodiments.

“Detect” refers to identifying the presence, absence or amount of the analyte to be detected.

By “disease” is meant any condition or disorder that damages or interferes with the normal function of a cell, tissue, or organ. Examples of diseases include inflammatory bowel disease (e.g., Crohn's disease, colitis), ETBF-induced colitis, colonic hyperplasia and tumor formation.

By “effective amount” is meant the amount of an agent required to ameliorate the symptoms of a disease relative to an untreated patient. The effective amount of active compound(s) used to practice the present invention for therapeutic treatment of a disease varies depending upon the manner of administration, the age, body weight, and general health of the subject. Ultimately, the attending physician or veterinarian will decide the appropriate amount and dosage regimen. Such amount is referred to as an “effective” amount.

The invention provides a number of targets that are useful for the development of highly specific drugs to treat or a disorder characterized by the methods delineated herein. In addition, the methods of the invention provide a facile means to identify therapies that are safe for use in subjects. In addition, the methods of the invention provide a route for analyzing virtually any number of compounds for effects on a disease described herein with high-volume throughput, high sensitivity, and low complexity.

By “fragment” is meant a portion of a cell, polypeptide or nucleic acid molecule. This portion contains, preferably, at least 10%, 20%, 30%, 40%, 50%, 60%, 70%, 80%, or 90% of the entire length of a reference nucleic acid molecule or polypeptide. A fragment may contain 10, 20, 30, 40, 50, 60, 70, 80, 90, or 100, 200, 300, 400, 500, 600, 700, 800, 900, or 1000 nucleotides or amino acids.

“Hybridization” means hydrogen bonding, which may be Watson-Crick, Hoogsteen or reversed Hoogsteen hydrogen bonding, between complementary nucleobases. For example, adenine and thymine are complementary nucleobases that pair through the formation of hydrogen bonds.

By “inhibitory nucleic acid” is meant a double-stranded RNA, siRNA, shRNA, or antisense RNA, or a portion thereof, or a mimetic thereof, that when administered to a mammalian cell results in a decrease (e.g., by 10%, 25%, 50%, 75%, or even 90-100%) in the expression of a target gene. Typically, a nucleic acid inhibitor comprises at least a portion of a target nucleic acid molecule, or an ortholog thereof, or comprises at least a portion of the complementary strand of a target nucleic acid molecule. For example, an inhibitory nucleic acid molecule comprises at least a portion of any or all of the nucleic acids delineated herein.

By “isolated polynucleotide” is meant a nucleic acid (e.g., a DNA) that is free of the genes which, in the naturally-occurring genome of the organism from which the nucleic acid molecule of the invention is derived, flank the gene. The term therefore includes, for example, a recombinant DNA that is incorporated into a vector; into an autonomously replicating plasmid or virus; or into the genomic DNA of a prokaryote or eukaryote; or that exists as a separate molecule (for example, a cDNA or a genomic or cDNA fragment produced by PCR or restriction endonuclease digestion) independent of other sequences. In addition, the term includes an RNA molecule that is transcribed from a DNA molecule, as well as a recombinant DNA that is part of a hybrid gene encoding additional polypeptide sequence.

By an “isolated polypeptide” is meant a polypeptide of the invention that has been separated from components that naturally accompany it. Typically, the polypeptide is isolated when it is at least 60%, by weight, free from the proteins and naturally-occurring organic molecules with which it is naturally associated. Preferably, the preparation is at least 75%, more preferably at least 90%, and most preferably at least 99%, by weight, a polypeptide of the invention. An isolated polypeptide of the invention may be obtained, for example, by extraction from a natural source, by expression of a recombinant nucleic acid encoding such a polypeptide; or by chemically synthesizing the protein. Purity can be measured by any appropriate method, for example, column chromatography, polyacrylamide gel electrophoresis, or by HPLC analysis.

By “marker” is meant any protein or polynucleotide having an alteration in expression level or activity that is associated with a disease or disorder.

As used herein, “obtaining” as in “obtaining an agent” includes synthesizing, purchasing, or otherwise acquiring the agent.

“Primer set” means a set of oligonucleotides that may be used, for example, for PCR. A primer set would consist of at least 2, 4, 6, 8, 10, 12, 14, 16 or more primers.

By “reduces” is meant a negative alteration of at least 10%, 25%, 50%, 75%, or 100%.

By “reference” is meant a standard or control condition.

A “reference sequence” is a defined sequence used as a basis for sequence comparison. A reference sequence may be a subset of or the entirety of a specified sequence; for example, a segment of a full-length cDNA or gene sequence, or the complete cDNA or gene sequence. For polypeptides, the length of the reference polypeptide sequence will generally be at least about 16 amino acids, preferably at least about 20 amino acids, more preferably at least about 25 amino acids, and even more preferably about 35 amino acids, about 50 amino acids, or about 100 amino acids. For nucleic acids, the length of the reference nucleic acid sequence will generally be at least about 50 nucleotides, preferably at least about 60 nucleotides, more preferably at least about 75 nucleotides, and even more preferably about 100 nucleotides or about 300 nucleotides or any integer thereabout or therebetween.

By “siRNA” is meant a double stranded RNA. Optimally, an siRNA is 18, 19, 20, 21, 22, 23 or 24 nucleotides in length and has a 2 base overhang at its 3′ end. These dsRNAs can be introduced to an individual cell or to a whole animal; for example, they may be introduced systemically via the bloodstream. Such siRNAs are used to downregulate mRNA levels or promoter activity.

By “specifically binds” is meant a compound or antibody that recognizes and binds a polypeptide of the invention, but which does not substantially recognize and bind other molecules in a sample, for example, a biological sample, which naturally includes a polypeptide of the invention.

Nucleic acid molecules useful in the methods of the invention include any nucleic acid molecule that encodes a polypeptide of the invention or a fragment thereof. Such nucleic acid molecules need not be 100% identical with an endogenous nucleic acid sequence, but will typically exhibit substantial identity. Polynucleotides having “substantial identity” to an endogenous sequence are typically capable of hybridizing with at least one strand of a double-stranded nucleic acid molecule. Nucleic acid molecules useful in the methods of the invention include any nucleic acid molecule that encodes a polypeptide of the invention or a fragment thereof. Such nucleic acid molecules need not be 100% identical with an endogenous nucleic acid sequence, but will typically exhibit substantial identity. Polynucleotides having “substantial identity” to an endogenous sequence are typically capable of hybridizing with at least one strand of a double-stranded nucleic acid molecule. By “hybridize” is meant pair to form a double-stranded molecule between complementary polynucleotide sequences (e.g., a gene described herein), or portions thereof, under various conditions of stringency. (See, e.g., Wahl, G. M. and S. L. Berger (1987) Methods Enzymol. 152:399; Kimmel, A. R. (1987) Methods Enzymol. 152:507).

For example, stringent salt concentration will ordinarily be less than about 750 mM NaCl and 75 mM trisodium citrate, preferably less than about 500 mM NaCl and 50 mM trisodium citrate, and more preferably less than about 250 mM NaCl and 25 mM trisodium citrate. Low stringency hybridization can be obtained in the absence of organic solvent, e.g., formamide, while high stringency hybridization can be obtained in the presence of at least about 35% formamide, and more preferably at least about 50% formamide. Stringent temperature conditions will ordinarily include temperatures of at least about 30° C., more preferably of at least about 37° C., and most preferably of at least about 42° C. Varying additional parameters, such as hybridization time, the concentration of detergent, e.g., sodium dodecyl sulfate (SDS), and the inclusion or exclusion of carrier DNA, are well known to those skilled in the art. Various levels of stringency are accomplished by combining these various conditions as needed. In a preferred: embodiment, hybridization will occur at 30° C. in 750 mM NaCl, 75 mM trisodium citrate, and 1% SDS. In a more preferred embodiment, hybridization will occur at 37° C. in 500 mM NaCl, 50 mM trisodium citrate, 1% SDS, 35% formamide, and 100 .μg/ml denatured salmon sperm DNA (ssDNA). In a most preferred embodiment, hybridization will occur at 42° C. in 250 mM NaCl, 25 mM trisodium citrate, 1% SDS, 50% formamide, and 200 μg/ml ssDNA. Useful variations on these conditions will be readily apparent to those skilled in the art.

For most applications, washing steps that follow hybridization will also vary in stringency. Wash stringency conditions can be defined by salt concentration and by temperature. As above, wash stringency can be increased by decreasing salt concentration or by increasing temperature. For example, stringent salt concentration for the wash steps will preferably be less than about 30 mM NaCl and 3 mM trisodium citrate, and most preferably less than about 15 mM NaCl and 1.5 mM trisodium citrate. Stringent temperature conditions for the wash steps will ordinarily include a temperature of at least about 25° C., more preferably of at least about 42° C., and even more preferably of at least about 68° C. In a preferred embodiment, wash steps will occur at 25° C. in 30 mM NaCl, 3 mM trisodium citrate, and 0.1% SDS. In a more preferred embodiment, wash steps will occur at 42 C in 15 mM NaCl, 1.5 mM trisodium citrate, and 0.1% SDS. In a more preferred embodiment, wash steps will occur at 68° C. in 15 mM NaCl, 1.5 mM trisodium citrate, and 0.1% SDS. Additional variations on these conditions will be readily apparent to those skilled in the art. Hybridization techniques are well known to those skilled in the art and are described, for example, in Benton and Davis (Science 196:180, 1977); Grunstein and Hogness (Proc. Natl. Acad. Sci., USA 72:3961, 1975); Ausubel et al. (Current Protocols in Molecular Biology, Wiley Interscience, New York, 2001); Berger and Kimmel (Guide to Molecular Cloning Techniques, 1987, Academic Press, New York); and Sambrook et al., Molecular Cloning: A Laboratory Manual, Cold Spring Harbor Laboratory Press, New York.

By “substantially identical” is meant a polypeptide or nucleic acid molecule exhibiting at least 50% identity to a reference amino acid sequence (for example, any one of the amino acid sequences described herein) or nucleic acid sequence (for example, any one of the nucleic acid sequences described herein). Preferably, such a sequence is at least 60%, more preferably 80% or 85%, and more preferably 90%, 95% or even 99% identical at the amino acid level or nucleic acid to the sequence used for comparison.

Sequence identity is typically measured using sequence analysis software (for example, Sequence Analysis Software Package of the Genetics Computer Group, University of Wisconsin Biotechnology Center, 1710 University Avenue, Madison, Wis. 53705, BLAST, BESTFIT, GAP, or PILEUP/PRETTYBOX programs). Such software matches identical or similar sequences by assigning degrees of homology to various substitutions, deletions, and/or other modifications. Conservative substitutions typically include substitutions within the following groups: glycine, alanine; valine, isoleucine, leucine; aspartic acid, glutamic acid, asparagine, glutamine; serine, threonine; lysine, arginine; and phenylalanine, tyrosine. In an exemplary approach to determining the degree of identity, a BLAST program may be used, with a probability score between e⁻³ and e⁻¹⁰⁰ indicating a closely related sequence.

By “subject” is meant a mammal, including, but not limited to, a human or non-human mammal, such as a bovine, equine, canine, ovine, or feline.

Ranges provided herein are understood to be shorthand for all of the values within the range. For example, a range of 1 to 50 is understood to include any number, combination of numbers, or sub-range from the group consisting 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50.

As used herein, the terms “treat,” treating,” “treatment,” and the like refer to reducing or ameliorating a disorder and/or symptoms associated therewith. It will be appreciated that, although not precluded, treating a disorder or condition does not require that the disorder, condition or symptoms associated therewith be completely eliminated.

Unless specifically stated or obvious from context, as used herein, the term “or” is understood to be inclusive. Unless specifically stated or obvious from context, as used herein, the terms “a”, “an”, and “the” are understood to be singular or plural.

Unless specifically stated or obvious from context, as used herein, the term “about” is understood as within a range of normal tolerance in the art, for example within 2 standard deviations of the mean. About can be understood as within 10%, 9%, 8%, 7%, 6%, 5%, 4%, 3%, 2%, 1%, 0.5%, 0.1%, 0.05%, or 0.01% of the stated value. Unless otherwise clear from context, all numerical values provided herein are modified by the term about.

The recitation of a listing of chemical groups in any definition of a variable herein includes definitions of that variable as any single group or combination of listed groups. The recitation of an embodiment for a variable or aspect herein includes that embodiment as any single embodiment or in combination with any other embodiments or portions thereof.

Any compositions or methods provided herein can be combined with one or more of any of the other compositions and methods provided herein.

### BRIEF DESCRIPTION OF THE DRAWINGS

FIGS. 1A-1D show that ETBF stimulated colonic inflammation and enhanced colonic tumor formation in Min mice. FIG. 1A provides micrographs showing methylene blue-stained representative samples of distal colons of sham control, non-enterotoxigenic B. fragilis (NTBF)-colonized and ETBF-colonized mice. Mice colonized with ETBF for 1-2 months showed thickened mucosal folds and excess tumors. FIG. 1B is a box- and -whisker plot showing the distribution of visible tumor numbers detected in sham control, NTBF- or ETBF-colonized mice at 4-6 weeks after inoculation. Tumor distributions are shown as box-and-whisker plots. n=14, 10 or 75 for sham control, NTBF or ETBF, respectively. FIG. 1C provides photomicrographs showing distal colon histopathology of sham control and NTBF-colonized mice at 4 weeks and ETBF-colonized mice at 1 week and 4 weeks after inoculation. Insets show gastrointestinal intraepithelial neoplasia (GIN) foci in sham and ETBF-colonized mice. FIG. 1D shows a linear regression analysis of histological scores of ETBF-colonized colons for inflammation and hyperplasia versus visible colon tumor formation or GIN foci. Error bars represent means±s.e.m

FIG. 2 shows that ETBF specifically activates Stat3 in the colons of Min mice. FIG. 2A is a Western blot analysis for activated Stat3 (pStat3) in colon samples of sham control Min mice or Min mice colonized with NTBF or ETBF for 2 days. Three individual mice are shown for each experimental condition. β-actin serves as a protein benchmark; protein concentrations per sample were equivalent (4.3-4.9 μg μl-1). The break in the gel (proximal colon) indicates that samples were run on separate gels analyzed in parallel for the same experiment. Data are representative of five sham-inoculated, six NTBF-colonized and six ETBF-colonized Min mice. FIG. 2B is a Western blot analysis for pStat proteins in colons of three ETBF-colonized Min mice. Positive controls for each pStat antibody are shown. β-actin served as a protein loading control. FIG. 2C provides micrographs showing an immunohistochemical analysis for pStat3 in distal colon of ETBF-colonized mice 4 weeks after inoculation compared to sham or NTBF-colonized mice. Arrows depict a subset of inflammatory cells in the lamina propria of ETBF-colonized mice that show pStat3 staining (FIG. 2E-b). Representative of two sham, four NTBF-colonized and seven ETBF-colonized Min mice. FIG. 2D provides micrographs showing an immunohistochemical analysis for pStat3 in a large colon tumor from an eight-week-old, sham-inoculated Min mouse and a similar-sized colon tumor in a Min mouse colonized with ETBF for 4 weeks. Arrows designate pStat3 staining of inflammatory cells in the interstitium.

FIG. 2E-a shows that ETBF induced predominantly pStat3 in colons of C57Bl/6 mice. pStat nuclear proteins were evaluated by western blot on nuclear extracts of colons of 3-4 ETBF-colonized C57Bl/6 mice. Positive and negative controls for each pStat protein are included for comparison. Equal amounts of nuclear protein were loaded in each lane. Actin further serves as a loading control. FIG. 2E-b shows pStat3 staining in infiltrating leukocytes of the colonic mucosa of an ETBF-colonized Min mouse. This provides an example of ETBF-colonized mucosa in which marked inflammatory infiltrates demonstrated pStat3 staining by IHC.

FIGS. 3A-3E are dot plots showing that ETBF, but not NTBF, induced IL-17-producing CD3⁺CD4⁺ T lymphocytes and γδ T lymphocytes in the colon lamina propria of Min and WT mice 1 week after NTBF or ETBF inoculation. FIG. 3A is an intracellular cytokine staining (ICS) for IL-17, IFN-γ and IL-4 in CD3⁺CD4⁺ T lymphocytes of Min mice. Dot plots are derived from the CD3⁺CD4⁺ gate. FIG. 3B shows an ICS for IL-17 in CD3⁺CD4⁺ and CD3⁺CD4⁻ lymphocytes from the lamina propria of ETBF-colonized Min mice. Dot plots are derived from CD3⁺ gate. FIG. 3C shows an ICS for IL-17 and IFN-γ in CD3⁺CD4⁺ and CD3⁺CD4⁻ T lymphocytes of C57BL/6 mice. Dot plots are derived from CD3⁺CD4⁺ and CD3⁺ gates. FIG. 3D shows an ICS for IL-17 in γδ T cells from the lamina propria of ETBFcolonized Min mice. Dot plots are derived from CD3⁺CD4⁻ gate. FIG. 3E shows ICS staining in CD3⁺CD4⁺ and CD3⁺CD4⁻ lymphocytes from WT and CD4 Stat3-KO C57BL/6 mice. Dot plots are derived from the CD3⁺ gate. Each panel is representative of at least three independent experiments except e (two independent experiments). The numbers inside the plots indicate the percentage of the cell population showing the quadrant characteristic.

FIG. 3F provides two dot plots showing the expression of γδ-TCR, αβ-TCR and CD4⁺ on the CD3⁺IL-17⁺ lymphocytes. CD3⁺CD8⁺ lymphocytes do not display ICS for IL-17. The dot plot is derived from CD3⁺ cells. The figure is representative of at least 3 independent experiments. Splenic cells isolated from Min mice colonized with ETBF for 1 wk and activated with PMA, ionomycin and Golgiplug™ for 5 hours were first stained for surface markers followed by intracellular staining for IFN-γ and IL-17.

FIG. 3G-a and 3G-b are dot plots showing results of an analysis carried out on isolated colonic lymphocytes from Min mice colonized with ETBF for 1 wk and activated 5 hours in vitro with PMA and ionomycin in the presence of Golgiplug™. FIG. 3G-a shows that CD3⁻ leukocytes do not display ICS for IL-17. Two right dot plots show the expression of γδ-TCR, αβ-TCR and CD4⁺ on the CD3⁺IL-17⁺ lymphocytes. FIG. 3G-b shows that CD3⁺CD8⁺ lymphocytes do not display ICS for IL-17. The dot plot is derived from CD3⁺ cells. The figure is representative of at least 3 independent experiments.

FIG. 3H is a box-and-whiskers plot showing the distribution of the cytokine data obtained in a qPCR analysis of Th17 pathway cytokines in the colonic mucosa of Min mice colonized with NTBF or ETBF for 1 wk. NT=NTBF-colonized and ET=ETBF-colonized mice. n=10−15 per condition except NT, IL-1β (n=4).

FIGS. 4A-4D show that blockade of IL-17 and IL-23R, but not IFN-γ, inhibited ETBF-induced colonic tumor formation in Min mice. FIG. 4A provides micrographs showing methylene blue-stained representative samples of distal colons of mice colonized with ETBF for 5 weeks and treated with IL-17 and IL-23R blocking antibodies or isotype control antibodies. FIG. 4B is a box- and whisker plot that depicts tumor number distribution in ETBF-colonized mice treated with isotype-matched antibodies (IgG+ET; experimental positive control) and ETBF-colonized mice treated with IL-17-(IL-17A+ET), IL-17- and IL-23R-(IL-17+IL-23R+ET) or IFN-γ-(IFN-γ+ET) blocking antibodies after 5 weeks. Sham-inoculated mice served as an experimental negative control. Top, n=24 for IgG+ET, 8 for IL-17+ET, 14 for IL-17+IL-23R+ET and 7 for sham. Bottom, n=9 for IgG+ET and 11 for IFN-γ+ET. FIG. 4C provides micrographs showing the histopathology of distal colon tumors in Min mice colonized with ETBF for 5 weeks and treated with isotype control antibodies (left) or IL-17- and IL-23R-blocking antibodies (right). Two representative mice of 24 (isotype control) or 14 (IL-17-blocking and IL-23R-blocking antibody treated) per treatment group are shown. FIG. 4D provides micrographs showing the histopathology of distal colon of Min mice colonized with ETBF for 1 week and treated with isotype control antibody (center) or IL-17- and IL-23R-blocking antibodies (right). Left image show the distal colon of a sham control Min mouse. Micrographs are representative of three sham control, five ETBF and isotype control antibody-treated and four ETBF, IL-17- and IL-23R-neutralizing antibody-treated mice.

FIG. 5 shows two box-and-whisker plots that depict a distribution of tumor numbers. CD4⁺, but not γδ⁺, T cell depletion inhibits tumor formation in ETBF-colonized Min mice. CD4⁺ T cells (CD4+ET) or γδ T cells (TCR γδ+ET) were depleted in ETBF-colonized mice using specific monoclonal antibodies and compared to ETBF-colonized mice treated in parallel with IgG isotype control antibodies (IgG+ET). Left, n=22 for IgG+ET and 24 for CD4+ET. Right, n=9 for IgG+ET and 11 for TCR γδ+ET.

FIGS. 6A and 6B are micrographs. Methylene blue-stained colon tumors were counted at 5 wks after bacterial inoculation and sized by microscopy. In panel (a), ETBF-colonized Min mice were treated with IL-17A and IL-23R neutralizing antibodies or isotype control antibodies. In panel (b), ETBF-colonized Min mice were treated with IL-17A neutralizing antibodies or isotype control antibodies.

FIG. 7 provides Western blots showing an analysis of activated Stat3 (pStat3). Actin serves as a loading control Sham and ETBF-colonized Min or C57Bl/6 mice were treated with IL-17 blocking antibody (IL-17 Ab+ET) or isotype control IgG antibody (IgG+ET) for 3 d followed by extraction of mucosal nuclear proteins.

FIG. 8 provides human data showing the presence of ETBF in pediatric patients with Crohn's disease.

FIG. 9 provides data showing that bft isoforms may differ in colon tumor induction.

### DETAILED DESCRIPTION OF THE INVENTION

The invention features compositions and methods that are useful for the treatment or prevention of inflammatory bowel disease and/or colon cancer.

The invention is based, at least in part, on the discovery that a human colonic bacterium, enterotoxigenic Bacteroides fragilis (ETBF) triggers colitis and strongly induces colonic tumors in multiple intestinal neoplasia (Min) mice. The results reported herein address the immunologic mechanisms of colonic carcinogenesis by a human colonic bacterium, enterotoxigenic Bacteroides fragilis (ETBF). ETBF secretes B. fragilis toxin (BFT), which causes human inflammatory diarrhea, but also asymptomatically colonizes a proportion of the human population. As reported in more detail below, whereas both ETBF and nontoxigenic B. fragilis (NTBF) chronically colonize mice, only ETBF triggers colitis and strongly induces colonic tumors in multiple intestinal neoplasia (Min) mice. ETBF induces robust, selective colonic signal transducer and activator of transcription-3 (Stat3) activation with colitis characterized by a selective T helper type 17 (TH17) response distributed between CD4⁺ T cell receptor-αβ(TCRαβ)⁺ and CD4-8-TCRγδ T cells. Antibody-mediated blockade of interleukin-17 (IL-17) as well as the receptor for IL-23, a key cytokine amplifying TH17 responses, inhibits ETBF-induced colitis, colonic hyperplasia and tumor formation. These results show a Stat3- and TH17-dependent pathway for inflammation-induced cancer by a common human commensal bacterium, providing not only a new mechanistic insight into human colon carcinogenesis, but also compositions and methods for preventing and treating human colon carcinogenesis, as well as inflammatory bowel disease.

The invention features compositions and methods useful for the diagnosis of inflammatory bowel disease, ETBF-induced colitis, colonic hyperplasia and/or colon carcinogenesis in a subject. These methods and compositions are based, in part, on the discovery that ETBF is present in biological samples (e.g., stool, urine, blood, serum, tissue) derived from a subject with colitis, colon cancer, or inflammatory bowel disease (e.g., Crohn's disease). In addition, the invention also provides methods and compositions for inhibiting the Stat3 pathway, which is induced in subjects having ETBF present in their colons. In other embodiments, the invention provides methods and compositions for disrupting or reducing interleukin-17 (IL-17) and/or IL-23 signaling to treat or prevent ETBF-induced colitis, colonic hyperplasia and tumor formation. Such compositions and methods are likely to be useful for the diagnosis, prevention and treatment of not only colon cancer, but also diseases associated with intestinal inflammation.

Crohn's disease (CD) and ulcerative colitis (UC) are chronic, idiopathic and clinically heterogeneous intestinal disorders collectively known as inflammatory bowel disease (IBD). Ulcerative colitis causes inflammation and ulcers in the top layer of the lining of the large intestine. In Crohn's disease, all layers of the intestine may be involved, and normal healthy bowel can be found between sections of diseased bowel. Complications of Crohn's disease include intestinal blockages, which may require surgery, as well as fistulas and fissures. To avoid such complications, it is important to get an accurate diagnosis early in the course of the illness to ensure that appropriate therapies are selected. Current diagnostic methods for inflammatory bowel disease are invasive and patients typically find these tests unpleasant. To improve patient compliance, diagnostic accuracy, and early and appropriate treatment, improved diagnostic methods are required.

### The Role of Infectious and Inflammatory Processes in Carcinogenesis

Infection-associated inflammatory processes are known to enhance carcinogenesis in the affected organs. In humans, for example, chronic hepatitis (hepatitis B virus or hepatitis C virus) leads to liver cancer, and chronic Helicobacter pylori infection leads to gastric cancer in some individuals. Increased cancer incidence is likewise found in experimental mouse models of both infection-induced and noninfectious inflammation. Conditional knockout mice have shown the importance of nuclear factor-κB (NF-κB) signaling not only in the epithelial cells that are the target of transformation, but also in myeloid cells that contribute to inflammation. How NF-κB-induced inflammatory processes drive carcinogenesis is unclear, although IL-6 seems to be pivotal (Naugler et al. Science 317, 121-124 (2007); Naugler et al., Trends Mol. Med. 14, 109-119 (2008)). IL-6 induces the procarcinogenic Stat3 pathway and transcriptionally activates proliferative, antiapoptotic and proangiogenic genes involved in cancer growth (Yu, Nat. Rev. Cancer 4, 97-105 (2004)). Stat3 signaling organizes the immune microenvironment of tumors to block generation of antitumor immune responses.

In contrast, little information exists on how adaptive immunity, particularly T cell responses, promote cancer. Given that T cell responses generate antitumor responses and more tumors occur in Rag^{-/-} mice and mice with defective interferon signaling (Dunn et al., Nat. Rev. Immunol. 6, 836-848 (2006), chronic innate inflammatory responses are postulated to promote carcinogenesis, whereas T cell-dependent responses are postulated to inhibit carcinogenesis.

Three effector pathways of T cell differentiation are now defined: TH1 responses promoted by Stat1 and Stat4 signaling, TH2 responses promoted by Stat6 signaling and TH17 responses promoted by Stat3 signaling. TH1 responses, driven by IL-12 and characterized by interferon-γ (IFN-γ) production, are typically anticarcinogenic, whereas little is known about the contribution of TH2 or TH17 responses to cancer.

The role of infectious and inflammatory processes in colon carcinogenesis is of considerable interest, as ˜1×10¹³ commensal bacteria colonize the colon, with inflammation resulting if colonic epithelial homeostasis is disrupted (Cho et al., Nat. Rev. Immunol. 8, 458-466 (2008)). Indeed, ulcerative colitis results in predictable development of colon cancer over time. The key role of inflammation in colonic carcinogenesis is emphasized by the diminished tumor formation in multiple intestinal neoplasia (Min) mice (heterozygous for the adenomatous polyposis coli (Apc) gene) when Toll-like receptor signaling is abrogated (Rakoff-Nahoum et al., Science 317, 124-127 (2007)). Because certain human enteric bacteria cause colitis, there is interest in whether any of them can promote colon cancer, analogous to the H. pylori promotion of stomach cancer. A molecular subgroup of B. fragilis, ETBF, that produces a metalloprotease toxin termed BFT was analyzed. ETBF causes acute inflammatory diarrheal disease in children and adults but also asymptomatically colonizes up to 20%-35% of adults.

To address mechanisms of ETBF-induced colitis and carcinogenesis Min mice were used. ETBF persistently colonized Min mice with a rapid, strong selective activation of Stat3, whereas the non-toxin-producing NTBF strain colonizes, but induces neither colitis nor Stat activation. ETBF colitis is characterized by a selective TH17 response with markedly increased colonic tumor formation. The TH17 response directly contributes to ETBF-induced tumorigenesis. These results demonstrate a Stat3- and TH17-dependent pathway for colon carcinogenesis induced by a common human commensal bacterium, thereby defining a distinct role for adaptive immunity in colon cancer pathogenesis.

B. fragilis Toxin (BFT)

Fragilysin or B. fragilis toxin (BFT), the toxin produced by ETBF, acts through cleavage of E-cadherin (Wu et al PNAS 95:14979=14984, 1998), resulting in disruption of the tight junction between colonic epithelial cells (CEC), induction of signaling through multiple pathways, including NF-kB, STAT3 and Wnt and activation of dendritic cells within the lamina propria, leading to colitis. As reported herein, mice that ingest ETBF exhibit an acute Th17-based colitis followed by a chronic colitis associated with longterm colonization and induction of colon cancer.

Three different variants of bft gene have been identified, bft-1, bft-2 and bft-3. Although several studies have investigated their distribution among ETBF strains from stool specimens, there are no published data to correlate disease outcome with isotype. As reported herein, an enrichment of bft-2 was observed in a cohort of pediatric Crohns disease patients. Large-scale epidemiological and clinical studies are hampered by the lack of standardized assays capable of detecting B. fragilis toxins in an isotype-specific fashion. There is currently no blood-based diagnostic test that could assess previous colonization with ETBF. Accordingly, the invention provides diagnostic and screening assays using both PCR and ELISA to detect isotype-specific bft in stool samples from patients with bowel inflammation disease and chronic colitis and an ELISA diagnostic test to detect serum IgG and IgA against BFT. These tests will allow for early detection and screening of both symptomatic and asymptomatic patients.

### Diagnostics

The presence of ETBF (e.g., by detection of bft-1, bft-2 and bft-3) in stool, urine, blood, serum, tissue or other biological samples is associated with inflammatory bowel disease (e.g., Crohn's disease, ulcerative colitis), ETBF-induced colitis, colonic hyperplasia and/or colon carcinogenesis. Accordingly, the present invention provides a number of diagnostic assays that are useful for the identification or characterization of inflammatory bowel disease and/or colon carcinogenesis.

In one embodiment, a patient having inflammatory bowel disease and/or colon carcinogenesis will show the presence of ETBF (e.g., bft-1, bft-2 and bft-3). The presence of an ETBF nucleic acid molecule in a biological sample from a subject is detected using methods known to the skilled artisan and described herein. The presence of an ETBF nucleic acid molecule or polypeptide in the biological sample (e.g., stool sample, blood sample) is a marker for inflammatory bowel disease and/or colon carcinogenesis or is indicative of an increased likelihood of developing inflammatory bowel disease and/or colon carcinogenesis. In another embodiment, an increase in the expression of an ETBF nucleic acid molecule relative to a reference is a marker for inflammatory bowel disease and/or colon carcinogenesis or is indicative of an increased likelihood of developing inflammatory bowel disease and/or colon carcinogenesis. An increase in an ETBF nucleic acid molecule is detected may be detected using quantitative PCR, real-time quantitative PCR (Q-rt-PCR)).

Primers used for amplification of an ETBF nucleic acid molecule, including but not limited to those primer sequences described herein, are useful in diagnostic methods of the invention. The primers of the invention embrace oligonucleotides of sufficient length and appropriate sequence so as to provide specific initiation of polymerization on a significant number of nucleic acids. Specifically, the term “primer” as used herein refers to a sequence comprising two or more deoxyribonucleotides or ribonucleotides, preferably more than three, and most preferably more than 8, which sequence is capable of initiating synthesis of a primer extension product, which is substantially complementary to a locus strand. The primer must be sufficiently long to prime the synthesis of extension products in the presence of the inducing agent for polymerization. The exact length of primer will depend on many factors, including temperature, buffer, and nucleotide composition. The oligonucleotide primer typically contains between 12 and 27 or more nucleotides, although it may contain fewer nucleotides. Primers of the invention are designed to be “substantially” complementary to each strand of the genomic locus to be amplified and include the appropriate G or C nucleotides as discussed above. This means that the primers must be sufficiently complementary to hybridize with their respective strands under conditions that allow the agent for polymerization to perform. In other words, the primers should have sufficient complementarity with the 5′ and 3′ flanking sequences to hybridize therewith and permit amplification of the genomic locus. While exemplary primers are provided herein, it is understood that any primer that hybridizes with the target sequences of the invention are useful in the method of the invention for detecting ETBF nucleic acid molecules.

In one embodiment, ETBF-specific primers amplify a desired genomic target using the polymerase chain reaction (PCR). The amplified product is then detected using standard methods known in the art. In one embodiment, a PCR product (i.e., amplicon) or real-time PCR product is detected by probe binding. In one embodiment, probe binding generates a fluorescent signal, for example, by coupling a fluorogenic dye molecule and a quencher moiety to the same or different oligonucleotide substrates (e.g., TaqMan® (Applied Biosystems, Foster City, Calif., USA), Molecular Beacons (see, for example, Tyagi et al., Nature Biotechnology 14(3):303-8, 1996), Scorpions® (Molecular Probes Inc., Eugene, Oreg., USA)). In another example, a PCR product is detected by the binding of a fluorogenic dye that emits a fluorescent signal upon binding (e.g., SYBR® Green (Molecular Probes)). Such detection methods are useful for the detection of an ETBF PCR product.

In another embodiment, hybridization with PCR probes that are capable of detecting an ETBF nucleic acid molecule, including genomic sequences, or closely related molecules, may be used to hybridize to a nucleic acid sequence derived from a patient having inflammatory bowel disease and/or colon carcinogenesis. The specificity of the probe determines whether the probe hybridizes to a naturally occurring sequence, allelic variants, or other related sequences. Hybridization techniques may be used to identify mutations indicative of inflammatory bowel disease and/or colon carcinogenesis, or may be used to monitor expression levels of these genes (for example, by Northern analysis (Ausubel et al., supra).

In yet another embodiment, humans may be diagnosed for a propensity to develop inflammatory bowel disease and/or colon carcinogenesis by detecting an ETBF nucleic acid molecule or polypeptide. In one embodiment, the ETBF nucleic acid molecule derived from a subject is compared to a reference ETBF sequence. The presence of a bft-2 variant or closely related variants indicates that the patient has or has a propensity to develop inflammatory bowel disease (e.g., Crohn's disease) and/or colon carcinogenesis.

In another approach, diagnostic methods of the invention are used to assay the presence or level of an ETBF polypeptide in a biological sample (e.g., stool, urine, blood, serum, tissue) relative to a reference (e.g., the level of ETBF polypeptide present in a sample obtained from a healthy control subject). In one embodiment, the presence or level of an ETBF polypeptide is detected using an antibody that specifically binds an ETBF polypeptide. Methods for making such antibodies are known in the art. Such antibodies are useful for the diagnosis of inflammatory bowel disease and/or colon carcinogenesis. Methods for measuring an antibody-ETBF complex include, for example, detection of fluorescence, luminescence, chemiluminescence, absorbance, reflectance, transmittance, birefringence or refractive index. Optical methods include microscopy (both confocal and non-confocal), imaging methods and non-imaging methods. Methods for performing these assays are readily known in the art. Useful assays include, for example, an enzyme immune assay (EIA) such as enzyme-linked immunosorbent assay (ELISA), a radioimmune assay (RIA), a Western blot assay, or a slot blot assay. These methods are also described in, e.g., Methods in Cell Biology: Antibodies in Cell Biology, volume 37 (Asai, ed. 1993); Basic and Clinical Immunology (Stites & Terr, eds., 7th ed. 1991); and Harlow & Lane, supra. Immunoassays can be used to determine the quantity of ETBF in a sample, where an increase in the level of the ETBF polypeptide is diagnostic of a patient having inflammatory bowel disease and/or colon carcinogenesis. Methods for generating such antibodies are known in the art and described herein below.

A diagnostic amount of an ETBF polypeptide or polynucleotide distinguishes between a sample obtained from a subject having an inflammatory bowel disease and/or colon carcinogenesis and a sample obtained from a control subject. The skilled artisan appreciates that the particular diagnostic amount used can be adjusted to increase sensitivity or specificity of the diagnostic assay depending on the preference of the diagnostician. In general, any significant increase (e.g., at least about 10%, 15%, 30%, 50%, 60%, 75%, 80%, or 90%) in the level of an ETBF polypeptide or nucleic acid molecule in the subject sample relative to a reference may be used to diagnose a inflammatory bowel disease and/or colon carcinogenesis. In one embodiment, the reference is the level of ETBF polypeptide or nucleic acid molecule present in a control sample obtained from a patient that does not have inflammatory bowel disease and/or colon carcinogenesis. In another embodiment, the reference is a baseline level of ETBF present in a biologic sample derived from a patient prior to, during, or after treatment for inflammatory bowel disease and/or colon carcinogenesis. In yet another embodiment, the reference is a standardized curve.

### Patient Monitoring

The disease state or treatment of a patient having inflammatory bowel disease and/or colon carcinogenesis can be monitored using the methods and compositions of the invention. In one embodiment, PCR of a stool sample is used to detect the expression of an ETBF nucleic acid molecule. In another embodiment, an ELISA is used to detect the expression of an ETBF polypeptide. Such monitoring may be useful, for example, in assessing the efficacy of a particular drug (e.g., antibiotic, therapeutic vaccine) in a patient. Therapeutics that reduce the expression of an ETBF nucleic acid molecule (e.g., bft-1, bft-2 and bft-3) or ETBF polypeptide or polypeptide variant, are taken as particularly useful in the invention.

### Types of Biological Samples

The level of an ETBF polypeptide or nucleic acid molecule can be measured in different types of biologic samples (e.g., stool, blood, tissue). In one embodiment, the biologic sample is a stool sample that includes nucleic acid molecules or polypeptides derived from commensal organisms. In another embodiment, the biologic sample is a biologic fluid sample (e.g., blood, blood plasma, serum, urine).

### ETBF Antibodies

ETBF can be detected in any standard immunological assay using an antibody that specifically binds ETBF or an ETBF variant. Antibodies are well known to those of ordinary skill in the science of immunology. As used herein, the term “antibody” means not only intact antibody molecules, but also fragments of antibody molecules that retain immunogen binding ability. Such fragments are also well known in the art and are regularly employed both in vitro and in vivo. Accordingly, as used herein, the term “antibody” means not only intact immunoglobulin molecules but also the well-known active fragments F(ab′)₂, and Fab. F(ab′)₂, and Fab fragments which lack the Fc fragment of intact antibody, clear more rapidly from the circulation, and may have less non-specific tissue binding of an intact antibody (Wahl et al., J. Nucl. Med. 24:316-325 (1983). The antibodies of the invention comprise whole native antibodies, bispecific antibodies; chimeric antibodies; Fab, Fab′, single chain V region fragments (scFv) and fusion polypeptides.

In one embodiment, an antibody that binds an ETBF polypeptide (e.g., ETBF, ETBF variant 1, 2, or 3) is monoclonal. Alternatively, the anti-ETBF antibody is a polyclonal antibody. The preparation and use of polyclonal antibodies are also known the skilled artisan. The invention also encompasses hybrid antibodies, in which one pair of heavy and light chains is obtained from a first antibody, while the other pair of heavy and light chains is obtained from a different second antibody. Such hybrids may also be formed using humanized heavy and light chains. Such antibodies are often referred to as “chimeric” antibodies.

In general, intact antibodies are said to contain “Fc” and “Fab” regions. The Fc regions are involved in complement activation and are not involved in antigen binding. An antibody from which the Fc′ region has been enzymatically cleaved, or which has been produced without the Fc′ region, designated an “F(ab′)₂” fragment, retains both of the antigen binding sites of the intact antibody. Similarly, an antibody from which the Fc region has been enzymatically cleaved, or which has been produced without the Fc region, designated an “Fab′” fragment, retains one of the antigen binding sites of the intact antibody. Fab′ fragments consist of a covalently bound antibody light chain and a portion of the antibody heavy chain, denoted “Fd.” The Fd fragments are the major determinants of antibody specificity (a single Fd fragment may be associated with up to ten different light chains without altering antibody specificity). Isolated Fd fragments retain the ability to specifically bind to immunogenic epitopes.

Antibodies can be made by any of the methods known in the art utilizing ETBF polypeptides (e.g., ETBF, ETBF variant 1, 2, or 3), or immunogenic fragments thereof, as an immunogen. One method of obtaining antibodies is to immunize suitable host animals with an immunogen and to follow standard procedures for polyclonal or monoclonal antibody production. Immunization of a suitable host can be carried out in a number of ways. Nucleic acid sequences encoding an ETBF polypeptide (e.g., ETBF, ETBF variant 1, 2, or 3), or immunogenic fragments thereof, can be provided to the host in a delivery vehicle that is taken up by immune cells of the host. The cells will in turn express the ETBF on the cell surface generating an immunogenic response in the host. Alternatively, nucleic acid sequences encoding an ETBF polypeptide (e.g., ETBF, ETBF variant 1, 2, or 3) can be administered as a DNA vaccine or immunogenic fragments thereof, can be expressed in vitro, followed by isolation of the ETBF and administration of the polypeptide to a suitable host in which antibodies are raised.

If desired, antibodies can be purified from a host. Antibody purification methods may include salt precipitation (for example, with ammonium sulfate), ion exchange chromatography (for example, on a cationic or anionic exchange column preferably run at neutral pH and eluted with step gradients of increasing ionic strength), gel filtration chromatography (including gel filtration HPLC), and chromatography on affinity resins such as protein A, protein G, hydroxyapatite, and anti-immunoglobulin.

Antibodies can be conveniently produced from hybridoma cells engineered to express the antibody. Methods of making hybridomas are well known in the art. The hybridoma cells can be cultured in a suitable medium, and spent medium can be used as an antibody source. Polynucleotides encoding the antibody of interest can in turn be obtained from the hybridoma that produces the antibody, and then the antibody may be produced synthetically or recombinantly from these DNA sequences. For the production of large amounts of antibody, it is generally more convenient to obtain an ascites fluid. The method of raising ascites generally comprises injecting hybridoma cells into an immunologically naive histocompatible or immunotolerant mammal, especially a mouse. The mammal may be primed for ascites production by prior administration of a suitable composition; e.g., Pristane.

Monoclonal antibodies (Mabs) produced by methods of the invention can be “humanized” by methods known in the art. “Humanized” antibodies are antibodies in which at least part of the sequence has been altered from its initial form to render it more like human immunoglobulins. Techniques to humanize antibodies are particularly useful when non-human animal (e.g., murine) antibodies are generated. Examples of methods for humanizing a murine antibody are provided in U.S. Pat. Nos. 4,816,567, 5,530,101, 5,225,539, 5,585,089, 5,693,762 and 5,859,205. ETBF specific antibodies may be generated against ETBF or an ETBF variant (BFT-1, -2, -3).

### ETBF Polypeptides and Analogs

Also included in the invention are ETBF polypeptides, including BFT-1, BFT-2, BFT-3, variants, or fragments thereof containing at least one alteration relative to a reference sequence. Such alterations include certain mutations, deletions, insertions, or post-translational modifications. The invention further includes analogs of any naturally-occurring polypeptide of the invention. Analogs can differ from naturally-occurring polypeptides of the invention by amino acid sequence differences, by post-translational modifications, or by both. Analogs of the invention will generally exhibit at least 85%, more preferably 90%, and most preferably 95% or even 99% identity with all or part of a naturally-occurring amino acid sequence of the invention. The length of sequence comparison is at least 10, 13, 15 amino acid residues, preferably at least 25 amino acid residues, and more preferably more than 35 amino acid residues. Again, in an exemplary approach to determining the degree of identity, a BLAST program may be used, with a probability score between e⁻³ and e⁻¹⁰⁰ indicating a closely related sequence. Modifications include in vivo and in vitro chemical derivatization of polypeptides, e.g., acetylation, carboxylation, phosphorylation, or glycosylation; such modifications may occur during polypeptide synthesis or processing or following treatment with isolated modifying enzymes. Analogs can also differ from the naturally-occurring polypeptides of the invention by alterations in primary sequence. These include genetic variants, both natural and induced (for example, resulting from random mutagenesis by irradiation or exposure to ethanemethylsulfate or by site-specific mutagenesis as described in Sambrook, Fritsch and Maniatis, Molecular Cloning: A Laboratory Manual (2d ed.), CSH Press, 1989, or Ausubel et al., supra). Also included are cyclized peptides, molecules, and analogs which contain residues other than L-amino acids, e.g., D-amino acids or non-naturally occurring or synthetic amino acids.

In addition to full-length polypeptides, the invention also includes fragments of any one of the polypeptides of the invention. As used herein, the term “a fragment” means at least 5, 10, 13, or 15 amino acids. In other embodiments a fragment is at least 20 contiguous amino acids, at least 30 contiguous amino acids, or at least 50 contiguous amino acids, and in other embodiments at least 60 to 80 or more contiguous amino acids. Fragments of the invention can be generated by methods known to those skilled in the art or may result from normal protein processing (e.g., removal of amino acids from the nascent polypeptide that are not required for biological activity or removal of amino acids by alternative mRNA splicing or alternative protein processing events).

### ETBF Polynucleotides

In general, the invention includes any nucleic acid sequence encoding an ETBF polypeptide (e.g., bft-1, bft-2 and bft-3). Such sequences are useful, for example, in generating a recombinant protein of the invention. Also included in the methods of the invention are any nucleic acid molecule containing at least one strand that hybridizes with such a nucleic acid sequence (e.g., an inhibitory nucleic acid molecule, such as a dsRNA, siRNA, shRNA, or antisense molecule). An isolated nucleic acid molecule can be manipulated using recombinant DNA techniques well known in the art. Thus, a nucleotide sequence contained in a vector in which 5′ and 3′ restriction sites are known, or for which polymerase chain reaction (PCR) primer sequences have been disclosed, is considered isolated, but a nucleic acid sequence existing in its native state in its natural host is not. An isolated nucleic acid may be substantially purified, but need not be. For example, a nucleic acid molecule that is isolated within a cloning or expression vector may comprise only a tiny percentage of the material in the cell in which it resides. Such a nucleic acid is isolated, however, as the term is used herein, because it can be manipulated using standard techniques known to those of ordinary skill in the art.

### Therapeutic Methods

Colonization with ETBF, which has been estimated to occur in between 5-30% of the general population according to various studies, induces both colitis and colon cancer. Therefore, specific antibiotic treatment (e.g., metronizole, doxycycline, clindamycin, imipenem, meropenem, beta-lactam/beta-lactamase inhibitor combinations, cefotetan, tigecycline, moxifloxacin and derivatives of these classes of antibiotics) or immunization against BFT is likely to be therapeutic. Accordingly, the invention provides methods for treating an asymptomatic subject identified as having an ETBF infection by administering to the subject an effective amount of an antibiotic that reduces the proliferation or survival of ETBF (e.g., metronizole, doxycycline). ETBF-colonized patients will likely benefit from frequent colon cancer screening and prophylactic vaccination against BFT may be warranted. This in turn will influence healthcare outcome and allow early intervention. Both the PCR and ELISA-based assays will make the diagnosis specific, sensitive, reproducible, safe and cost effective. These tests will also allow large epidemiological studies to determine the prevalence of the 3 isotypes produced by ETBF.

Once the presence of ETBF (BFT-1, -2, -3) is identified in a subject, the subject is identified as having or having a propensity to develop ETBF-induced colitis, colonic hyperplasia and tumor formation. In one embodiment, asymptomatic subjects are treated with antibiotics (e.g., metronizole, doxycycline, clindamycin, imipenem, meropenem, beta-lactam/beta-lactamase inhibitor combinations, cefotetan, tigecycline, moxifloxacin and derivatives of these classes of antibiotics) to reduce the survival or proliferation of enterotoxigenic B. fragilis in the subject. In another embodiment, such subjects are treated with an immunogenic composition sufficient to generate an immune response against enterotoxigenic B. fragilis to treat or prevent an ETBF infection.

Subjects identified as having an ETBF infection who also display symptoms of inflammatory bowel disease are treated more aggressively than asymptomatic subjects. Such subjects should be treated with an antibiotic and/or a STAT3 inhibitor and/or an ETBF immunogenic composition. Subjects identified as having an ETBF, who display symptoms of inflammatory bowel disease, and who also display colonic hyperplasia or tumors are identified as in need of the most aggressive treatment. Such subjects are generally treated with a STAT3 inhibitor, an antibiotic, and conventional treatments for colon cancer. Their treatment may also include administration of a therapeutic ETBF vaccine.

STAT3 inhibitors include agents that reduce the expression or activity of STAT3. STAT3 inhibitors include, but are not limited to inhibitory nucleic acids that reduce STAT3 transcription or translation (e.g., antisense, siRNA, shRNA targeting STAT3); AG 490 (Jaleel et al. (2004) Biochemistry 43, 8247; Eriksen et al. (2001) Leukemia 15, 787; Kirken et al. (1999) Leukoc. Biol. 65, 891; Nielsen et al. (1997) Proc. Natl. Acad. Sci. USA 94, 6764; Meydan et al. (1996) Nature 379, 645; Gazit et al. (1991) J. Med. Chem. 34, 1896; A. Levitzki (1990) Biochem. Pharmacol. 40, 913); Cucurbitacin I (Blaskovich et al. (2003) Cancer Res. 63, 1270); STAT3 Inhibitor Peptide (Turkson et al. (2001) J. Biol. Chem. 276, 45443); Flavopiridol (Lee et al. (2006) Mol Cancer Ther. 5, 138-148); and Piceatannol (Alas et al. (2003) Clinical Cancer Research 9, 316-326). Other STAT3 inhibitors include, NSC 74859 as described by Lin et al., Oncogene 28, 961-972, 2009; 531-M2001 as described by Siddiquee et al. ACS Chem. Biol. 2007; 2(12): 787-98; and Stattic as described by Schust et al., Chemistry & Biology, Volume 13, Issue 11, 1235-1242, 1 Nov. 2006. Examples of compounds in preclinical or clinical use, include, e.g., AP23573, AP23841, CCI-779, and RAD001. Stat3 inibitors are also described at 20100041685, 20090069420, 20070060521, 20070010428, and 20040175369. Each of the aforementioned publications is hereby incorporated by reference in its entirety.

As reported herein, antibody-mediated blockade of interleukin-17 (IL-17) and the receptor for IL-23, a key cytokine amplifying TH17 responses, inhibited ETBF-induced colitis, colonic hyperplasia and tumor formation. Accordingly, the invention provides methods for treating or preventing inflammatory bowel disease or colon carcinogenesis in a subject by administering to the subject an effective amount of an agent that reduces IL-17 biological activity (e.g., an antibody that specifically binds IL-17 and blocks IL-17 binding to an IL-17 receptor, soluble form of the IL-17R used as a decoy or a drug that interferes with IL-17R signaling). In other embodiments, the invention provides methods for treating or preventing inflammatory bowel disease or colon carcinogenesis in a subject by administering to the subject an effective amount of an agent that reduces IL-23 binding to an IL-23 receptor. In one embodiment, the agent is an antibody that specifically blocks IL-23 binding to the IL-23 receptor, soluble form of the IL-23R used as a decoy or a drug that interferes with IL-23R signaling. Such methods may be used alone or in combination with any other therapeutic method delineated herein.

### Therapeutic and Prophylactic ETBF Vaccines

The invention also encompasses vaccine formulations comprising a killed or attenuated enterotoxigenic B. fragilis cell, bacterial protein (e.g., BFT-1, -2, or -3 protein) or immunogenic fragment thereof, and/or ETBF nucleic acid molecule (e.g., bft-1, bft-2 and bft-3) or immunogenic fragment thereof. Generally, bacteria, protein and/or polynucleotide immunogens of the invention are administered in an effective amount or quantity (as described herein) sufficient to stimulate an immune response against one or more strains of a bacteria described here, for example, enterotoxigenic B. fragilis or an isotype thereof. Preferably, administration of the bacteria, ETBF polypeptide and/or polynucleotide of the invention elicits immunity against enterotoxin B. fragilis. Typically, the dose can be adjusted within this range based on, e.g., age, physical condition, body weight, sex, diet, time of administration, and other clinical factors. The prophylactic or therapeutic vaccine formulation is systemically administered, e.g., by subcutaneous or intramuscular injection using a needle and syringe, or a needle-less injection device. In one embodiment, the vaccine is administered as an oral vaccine comprising live attenuated ETBF.

In certain cases, stimulation of immunity with a single dose is preferred, however additional dosages can be also be administered, by the same or different route, to achieve the desired effect. In neonates and infants, for example, multiple administrations may be required to elicit sufficient levels of immunity. Administration can continue at intervals throughout childhood, as necessary to maintain sufficient levels of protection against infections. Similarly, adults who are particularly susceptible to repeated or serious infections, such as, for example, the elderly, and individuals with compromised immune systems may require multiple immunizations to establish and/or maintain protective immune responses. Levels of induced immunity can be monitored, for example, by measuring amounts of neutralizing secretory and serum antibodies, and dosages adjusted or vaccinations repeated as necessary to elicit and maintain desired levels of protection.

Prime Boost

The present methods also include a variety of prime-boost regimens. In these methods, one or more priming immunizations is followed by one or more boosting immunizations. The actual immunogenic composition can be the same or different for each immunization and the route, and formulation of the immunogens can also be varied. For example, the prime-boost regimen can include administration of an immunogenic composition comprising a bacteria, bacterial polypeptide or bacterial polynucleotide. Vaccines and/or antigenic formulations of the invention may also be administered on a dosage schedule, for example, an initial administration of the vaccine composition with subsequent booster administrations. In particular embodiments, a second dose of the composition is administered anywhere from two weeks to one year, preferably from about 1, about 2, about 3, about 4, about 5 to about 6 months, after the initial administration. Additionally, a third dose may be administered after the second dose and from about three months to about two years, or even longer, preferably about 4, about 5, or about 6 months, or about 7 months to about one year after the initial administration. The third dose may be optionally administered when no or low levels of specific immunoglobulins are detected in the serum and/or urine or mucosal secretions of the subject after the second dose.

The dosage of the pharmaceutical formulation can be determined readily by the skilled artisan, for example, by first identifying doses effective to elicit a prophylactic or therapeutic immune response, e.g., by measuring the serum titer of enterotoxigenic B. fragilis specific immunoglobulins or by measuring the inhibitory ratio of antibodies in serum samples, or urine samples, or mucosal secretions. The dosages can be determined from animal studies. A non-limiting list of animals used to study the efficacy of vaccines include the guinea pig, hamster, ferrets, chinchilla, mouse and cotton rat, and non-human primates. Most animals are not natural hosts to infectious agents but can still serve in studies of various aspects of the disease. For example, any of the above animals can be dosed with a vaccine candidate, e.g. ETBF polypeptide of the invention, to partially characterize the immune response induced, and/or to determine if any neutralizing antibodies have been produced. In addition, human clinical studies can be performed to determine the preferred effective dose for humans by a skilled artisan. Such clinical studies are routine and well known in the art. The precise dose to be employed will also depend on the route of administration. Effective doses may be extrapolated from dose-response curves derived from in vitro or animal test systems.

The bacteria, polypeptide or polynucleotide immunogenic vaccines of the invention can also be formulated with “immune stimulators.” These are the body's own chemical messengers (cytokines) to increase the immune system's response. Immune stimulators include, but not limited to, various cytokines, lymphokines and chemokines with immunostimulatory, immunopotentiating, and pro-inflammatory activities, such as interleukins (e.g., IL-1, IL-2, IL-3, IL-4, IL-12, IL-13); growth factors (e.g., granulocyte-macrophage (GM)-colony stimulating factor (CSF)); and other immunostimulatory molecules, such as macrophage inflammatory factor, Flt3 ligand, B7.1; B7.2, etc. The immunostimulatory molecules can be administered in the same formulation as the vaccine, or can be administered separately. Either the protein or an expression vector encoding the protein can be administered to produce an immunostimulatory effect.

### Methods of Delivery

The enterotoxigenic B. fragilis polypeptides and polynucleotides are useful for preparing compositions that stimulate an immune response. Such compositions are useful for the treatment or prevention or a bacterial infection (e.g., a enterotoxigenic B. fragilis infection of the colon). Both mucosal and cellular immunity may contribute to immunity to infectious agents and disease. In one embodiment, the invention encompasses a method of inducing immunity to a bacterial infection, for example enterotoxigenic B. fragilis infection in a subject, by administering to the subject a composition comprising a enterotoxigenic B. fragilis cell, ETBF polypeptide, or ETBF polynucleotide (e.g., bft-1, bft-2 and bft-3).

The invention also provides a method to induce immunity to a bacterial infection or at least one symptom thereof in a subject, comprising administering at least one effective dose of a enterotoxigenic B. fragilis killed or attenuated vaccine, an ETBF polypeptide or ETBF polynucleotide. In one embodiment, the method comprises inducing immunity to a bacterial infection, e.g. enterotoxigenic B. fragilis infection or at least one symptom thereof by administering the formulation in multiple doses.

ETBF nucleic acid molecules and/or ETBF polypeptides of the invention can induce substantial immunity in a vertebrate (e.g. a human) when administered to the vertebrate. The substantial immunity results from an immune response against enterotoxigenic B. fragilis that protects or ameliorates infection or at least reduces a symptom of infection in the vertebrate. In some instances, if the vertebrate is infected, the infection will be asymptomatic. The response may be not a fully protective response. In this case, if the vertebrate is infected with an infectious agent, the vertebrate will experience reduced symptoms or a shorter duration of symptoms compared to a non-immunized vertebrate.

As mentioned above, the immunogenic compositions of the invention prevent or reduce at least one symptom of an enterotoxigenic B. fragilis infection in a subject (e.g., a reduction in ETBF-induced colitis, colonic hyperplasia and/or tumor formation). A reduction in a symptom may be determined subjectively or objectively, e.g., self assessment by a subject, by a clinician's assessment or by conducting an appropriate assay or measurement (e.g. tumor size, number), including, e.g., a quality of life assessment, a slowed progression of bacterial infection or additional symptoms, a reduced severity of colitis symptoms or a suitable assays (e.g. antibody titer). The objective assessment comprises both animal and human assessments.

### Immunogenic Compositions

The invention provides compositions and methods for inducing an immunological response in a subject, particularly a human, which involves inoculating the subject with a killed or attenuated enterotoxigenic B. fragilis cell, ETBF polypeptides, ETBF polynucleotides or fragments thereof, or a combination thereof, in a suitable carrier for the purpose of inducing or enhancing an immune response. In one embodiment, an immune response protects the subject from a enterotoxigenic B. fragilis infection or from ETBF-induced colitis, colonic hyperplasia and tumor formation. The administration of this immunological composition may be used either therapeutically in subjects already experiencing a enterotoxigenic B. fragilis infection, or may be used prophylactically to prevent or reduce ETBF-induced colitis, colonic hyperplasia and tumor formation.

The preparation of immunogenic compositions and vaccines is known to one skilled in the art. In one embodiment, the vaccine comprises one or more ETBF polypeptides (BFT-1, -2, -3), or fragments thereof. In another embodiment, the invention provides an expression vector encoding one or more enterotoxigenic B. fragilis polypeptides or fragments thereof or variants thereof. Such an immunogenic composition is delivered in vivo in order to induce or enhance an immunological response in a subject.

Typically vaccines are prepared in an injectable form, either as a liquid solution or as a suspension. Solid forms suitable for injection may also be prepared as emulsions, or with the polypeptides encapsulated in liposomes. Vaccine antigens are usually combined with a pharmaceutically acceptable carrier, which includes any carrier that does not induce the production of antibodies harmful to the subject receiving the carrier. Suitable carriers typically comprise large macromolecules that are slowly metabolized, such as proteins, polysaccharides, polylactic acids, polyglycolic acids, polymeric amino acids, amino acid copolymers, lipid aggregates, and inactive virus particles. Such carriers are well known to those skilled in the art. These carriers may also function as adjuvants.

The ETBF polypeptide comprising may be administered in combination with an adjuvant. Adjuvants are immunostimulating agents that enhance vaccine effectiveness. If desired, the enterotoxigenic B. fragilis killed or attenuated cells, ETBF polypeptides, polynucleotides, or fragments or variants thereof are administered in combination with an adjuvant that enhances the effectiveness of the immune response generated against the antigen of interest. Effective adjuvants include, but are not limited to, aluminum salts such as aluminum hydroxide and aluminum phosphate, muramyl peptides, bacterial cell wall components, saponin adjuvants, and other substances that act as immunostimulating agents to enhance the effectiveness of the composition.

Immunogenic compositions, pharmaceutically acceptable carrier and adjuvant, also typically contain diluents, such as water, saline, glycerol, ethanol. Auxiliary substances may also be present, such as wetting or emulsifying agents, pH buffering substances, and the like. Proteins may be formulated into the vaccine as neutral or salt forms. The immunogenic compositions are typically administered parenterally, by injection; such injection may be either subcutaneously or intramuscularly. Additional formulations are suitable for other forms of administration, such as by suppository or orally. Oral compositions may be administered as a solution, suspension, tablet, pill, capsule, or sustained release formulation.

Immunogenic compositions are administered in a manner compatible with the dose formulation. The immunogenic composition comprises an immunologically effective amount of the immunogens and other previously mentioned components. By an immunologically effective amount is meant a single dose, or a composition administered in a multiple dose schedule, that is effective for the treatment or prevention of an infection. The dose administered will vary, depending on the subject to be treated, the subject's health and physical condition, the capacity of the subject's immune system to produce antibodies, the degree of protection desired, and other relevant factors. Precise amounts of the active ingredient required will depend on the judgment of the practitioner, but typically range between 5 μg to 250 μg of antigen per dose. The invention provides a ETBF polypeptide (e.g., BFT-1, BFT-2, BFT-3, as well as other isoforms) or polynucleotide for use in treating or preventing a enterotoxigenic B. fragilis infection (e.g., bft-1, bft-2 and bft-3). In particular, the present invention provides methods of treating bacterial diseases and/or disorders or symptoms thereof which comprise administering a therapeutically effective amount of a pharmaceutical composition comprising a ETBF nucleic acid molecule to a subject (e.g., a mammal such as a human). Thus, one embodiment is a method of treating a subject suffering from or susceptible to a bacterial infection, bacterial disease or disorder or symptom thereof. The method includes the step of administering to the mammal a therapeutic or prophylactic amount of a compound herein sufficient to treat the disease or disorder or symptom thereof, under conditions such that the disease or disorder is prevented or treated.

The methods herein include administering to the subject (including a subject identified as in need of such treatment) an effective amount of a compound described herein, or a composition described herein to produce such effect. Identifying a subject in need of such treatment can be in the judgment of a subject or a health care professional and can be subjective (e.g. opinion) or objective (e.g. measurable by a test or diagnostic method).

The therapeutic methods of the invention (which include prophylactic treatment) in general comprise administration of a therapeutically effective amount of the agents herein, such as a ETBF polypeptide or polynucleotide of a formulae herein to a subject (e.g., animal, human) in need thereof, including a mammal, particularly a human. Such treatment will be suitably administered to subjects, particularly humans, suffering from, having, susceptible to, or at risk for ETBF-induced colitis, colonic hyperplasia and tumor formation. Determination of those subjects “at risk” can be made by any objective or subjective determination by a diagnostic test or opinion of a subject or health care provider (e.g., genetic test, enzyme or protein marker, Marker (as defined herein), family history, and the like). The agents herein may be also used in the treatment of any other disorders in which a enterotoxigenic B. fragilis infection may be implicated.

In one embodiment, the invention provides a method of monitoring treatment progress. The method includes the step of determining a level of diagnostic marker (Marker) (e.g., any target delineated herein modulated by a compound herein, a protein or indicator thereof, etc.) or diagnostic measurement (e.g., screen, assay) in a subject suffering from or susceptible to a disorder or symptoms thereof associated with ETBF, in which the subject has been administered a therapeutic amount of a compound herein sufficient to treat the disease or symptoms thereof. The level of Marker determined in the method can be compared to known levels of Marker in either healthy normal controls or in other afflicted patients to establish the subject's disease status. In preferred embodiments, a second level of Marker in the subject is determined at a time point later than the determination of the first level, and the two levels are compared to monitor the course of disease or the efficacy of the therapy. In certain preferred embodiments, a pre-treatment level of Marker in the subject is determined prior to beginning treatment according to this invention; this pre-treatment level of Marker can then be compared to the level of Marker in the subject after the treatment commences, to determine the efficacy of the treatment.

### Screening Assays

As reported herein, the presence of ETBF nucleic acid molecule or polypeptide in a biological sample is associated with inflammatory bowel disease and/or colon carcinogenesis. ETBF induces robust, selective colonic signal transducer and activator of transcription-3 (Stat3) activation with colitis characterized by a selective T helper type 17 (TH17) response distributed between CD4⁺ T cell receptor-αβ(TCRαβ)⁺ and CD4-8-TCRγδ⁺ T cells. Accordingly, agents that reduce the expression or activity of a STAT-3 polypeptide, variant, or fragment thereof are useful for the treatment or prevention of inflammatory bowel disease and/or colon carcinogenesis. Antibody-mediated blockade of interleukin-17 (IL-17) as well as the receptor for IL-23, a key cytokine amplifying TH17 responses, inhibits ETBF-induced colitis, colonic hyperplasia and tumor formation. Accordingly, agents that reduce or inhibit IL-17 and/or IL-23 activity are also useful for the treatment or prevention of ETBF-induced colitis, colonic hyperplasia and tumor formation. In still other embodiments, agents that reduce the expression or activity of ETBF or that reduce enterotoxigenic B. fragilis proliferation or survival are also useful for the treatment or prevention of inflammatory bowel disease and/or colon carcinogenesis.

Any number of methods are available for carrying out screening assays to identify such compounds. In one approach, candidate compounds are identified that specifically bind to and reduce the activity of a polypeptide of the invention (e.g., ETBF, STAT-3, IL-17, IL-23). Methods of assaying such biological activities are known in the art and are described herein. The efficacy of such a candidate compound is dependent upon its ability to interact with a polypeptide of interest, variant, or fragment. Such an interaction can be readily assayed using any number of standard binding techniques and functional assays (e.g., those described in Ausubel et al., supra). In other embodiments, a candidate compound may be tested in vitro for its activity (e.g., its ability to reduce B. fragilis cell proliferation or survival, reduce ETBF, STAT-3, IL-17, and/or IL-23 expression or biological activity).

Potential agents that reduce the proliferation of B. fragilis or that antagonize a polypeptide of the invention include organic molecules, peptides, peptide mimetics, polypeptides, nucleic acid molecules and antibodies that bind to a nucleic acid sequence or polypeptide of the invention and thereby inhibit or extinguish its activity. Potential antagonists also include small molecules that bind to the polypeptide of interest (e.g., ETBF, STAT-3, IL-17, IL-23) thereby preventing binding to cellular molecules with which the polypeptide normally interacts, such that the normal biological activity of the polypeptide is reduced or inhibited. Small molecules of the invention preferably have a molecular weight below 2,000 daltons, more preferably between 300 and 1,000 daltons, and most preferably between 400 and 700 daltons. It is preferred that these small molecules are organic molecules.

In one particular example, a candidate compound that binds to a polypeptide of interest (e.g., ETBF, STAT-3, IL-17, IL-23), variant, or fragment thereof may be identified using a chromatography-based technique. For example, a recombinant polypeptide of the invention may be purified by standard techniques from cells engineered to express the polypeptide (e.g., those described above) and may be immobilized on a column. A solution of candidate compounds is then passed through the column, and a compound specific for the polypeptide is identified on the basis of its ability to bind to the polypeptide and be immobilized on the column. To isolate the compound, the column is washed to remove non-specifically bound molecules, and the compound of interest is then released from the column and collected.

Similar methods may be used to isolate an agent bound to a polypeptide microarray. Compounds isolated by this method (or any other appropriate method) may, if desired, be further purified (e.g., by high performance liquid chromatography). In addition, these candidate compounds may be tested for their ability to alter the biological activity of a polypeptide of interest (e.g., ETBF, STAT-3, IL-17, IL-23), to inhibit or reduce the proliferation of B. fragilis, or to prevent, reduce, or ameliorate ETBF-induced colitis, colonic hyperplasia and/or tumor formation.

Compounds that are identified as binding to a polypeptide of the invention (e.g., ETBF, STAT-3, IL-17, IL-23) with an affinity constant less than or equal to 10 mM are considered particularly useful in the invention. Alternatively, any in vivo protein interaction detection system, for example, any two-hybrid assay may be utilized to identify compounds that interact with such a polypeptide. Interacting compounds isolated by this method (or any other appropriate method) may, if desired, be further purified (e.g., by high performance liquid chromatography). Compounds isolated by any approach described herein may be used as therapeutics to treat or prevent inflammatory bowel disease and/or colon carcinogenesis in a human patient.

In addition, compounds that inhibits the expression of a nucleic acid molecule of interest (e.g., a nucleic acid molecule encoding ETBF, STAT-3, IL-17, IL-23) whose expression is increased in a patient having a neoplasia are also useful in the methods of the invention. Any number of methods are available for carrying out screening assays to identify new candidate compounds that reduce the expression of such nucleic acid molecules. In one working example, candidate compounds are added at varying concentrations to the culture medium of cultured cells. Gene expression is then measured, for example, by microarray analysis, Northern blot analysis (Ausubel et al., supra), or RT-PCR, using any appropriate fragment prepared from the nucleic acid molecule as a hybridization probe. The level of gene expression in the presence of the candidate compound is compared to the level measured in a control culture medium lacking the candidate molecule. A compound that reduces the expression of a gene of interest (e.g., ETBF, STAT-3, IL-17, IL-23), or a functional equivalent thereof, is considered useful in the invention; such a molecule may be used, for example, as a therapeutic to treat or prevent an inflammatory bowel disease and/or colon carcinogenesis in a human patient.

In another approach, the effect of an agent is measured at the level of polypeptide production to identify those that reduce the level of a polypeptide of interest (e.g., ETBF, STAT-3, IL-17, IL-23). The level of polypeptide can be assayed using any standard method. Standard immunological techniques include Western blotting or immunoprecipitation with an antibody specific for an ETBF polypeptide (e.g., ETBF, ETBF variant 1, 2, or 3). For example, immunoassays may be used to detect or monitor the expression of at least one of the polypeptides of the invention in an organism. Polyclonal or monoclonal antibodies (produced as described above) that are capable of binding to such a polypeptide may be used in any standard immunoassay format (e.g., ELISA, Western blot, or RIA assay) to measure the level of the polypeptide. In some embodiments, a compound that promotes a decrease in the expression or biological activity of the polypeptide is considered particularly useful. Again, such a molecule may be used, for example, as a therapeutic to delay, ameliorate, or treat inflammatory bowel disease and/or colon carcinogenesis in a human patient.

In another embodiment, a nucleic acid described herein (e.g., NFκB, MAPK, Wnt, STAT-3,) is expressed as a transcriptional or translational fusion with a detectable reporter, and expressed in an isolated cell (e.g., epithelial cell derived from colon or intestine such as HT29, HT29/C1, Caco-2, T84 or other immortalized intestinal epithelial cell line) under the control of a heterologous promoter, such as an inducible promoter. The cell expressing the fusion protein is then contacted with a candidate compound, and the expression of the detectable reporter in that cell is compared to the expression of the detectable reporter in an untreated control cell. A candidate compound that reduces the expression of the detectable reporter is a compound that is useful for the treatment of inflammatory bowel disease and/or colon carcinogenesis. In one embodiment, the compound decreases the expression of the reporter.

Each of the DNA sequences listed herein may also be used in the discovery and development of a therapeutic compound for the treatment of inflammatory bowel disease and/or colon carcinogenesis. The encoded protein, upon expression, can be used as a target for the screening of drugs. Additionally, the DNA sequences encoding the amino terminal regions of the encoded protein or Shine-Delgarno or other translation facilitating sequences of the respective mRNA can be used to construct sequences that promote the expression of the coding sequence of interest. Such sequences may be isolated by standard techniques (Ausubel et al., supra).

The invention also includes novel compounds identified by the above-described screening assays. Optionally, such compounds are characterized in one or more appropriate animal models to determine the efficacy of the compound for the treatment or prevention of ETBF-induced colitis, colonic hyperplasia and tumor formation. Desirably, characterization in an animal model can also be used to determine the toxicity, side effects, or mechanism of action of treatment with such a compound. Furthermore, novel compounds identified in any of the above-described screening assays may be used for the treatment or prevention of ETBF-induced colitis, colonic hyperplasia and tumor formation in a subject. Such compounds are useful alone or in combination with other conventional therapies known in the art.

### Test Compounds and Extracts

In general, compounds capable of inhibiting ETBF-induced colitis, colonic hyperplasia and tumor formation by altering the expression or biological activity of a polypeptide of interest (e.g., BFT-1, BFT-2, BFT-3, STAT-3, IL-17, IL-23), variant, or fragment thereof are identified from large libraries of either natural product or synthetic (or semi-synthetic) extracts or chemical libraries according to methods known in the art. Numerous methods are also available for generating random or directed synthesis (e.g., semi-synthesis or total synthesis) of any number of chemical compounds, including, but not limited to, saccharide-, lipid-, peptide-, and nucleic acid-based compounds. Synthetic compound libraries are commercially available from Brandon Associates (Merrimack, N.H.) and Aldrich Chemical (Milwaukee, Wis.). Alternatively, libraries of natural compounds in the form of bacterial, fungal, plant, and animal extracts are commercially available from a number of sources, including Biotics (Sussex, UK), Xenova (Slough, UK), Harbor Branch Oceangraphics Institute (Ft. Pierce, Fla.), and PharmaMar, U.S.A. (Cambridge, Mass.).

In one embodiment, test compounds of the invention are present in any combinatorial library known in the art, including: biological libraries; peptoid libraries (libraries of molecules having the functionalities of peptides, but with a novel, non-peptide backbone which are resistant to enzymatic degradation but which nevertheless remain bioactive; see, e.g., Zuckermann, R. N. et al., J. Med. Chem. 37:2678-85, 1994); spatially addressable parallel solid phase or solution phase libraries; synthetic library methods requiring deconvolution; the ‘one-bead one-compound’ library method; and synthetic library methods using affinity chromatography selection. The biological library and peptoid library approaches are limited to peptide libraries, while the other four approaches are applicable to peptide, non-peptide oligomer or small molecule libraries of compounds (Lam, Anticancer Drug Des. 12:145, 1997).

Examples of methods for the synthesis of molecular libraries can be found in the art, for example in: DeWitt et al., Proc. Natl. Acad. Sci. U.S.A. 90:6909, 1993; Erb et al., Proc. Natl. Acad. Sci. USA 91:11422, 1994; Zuckermann et al., J. Med. Chem. 37:2678, 1994; Cho et al., Science 261:1303, 1993; Carrell et al., Angew. Chem. Int. Ed. Engl. 33:2059, 1994; Carell et al., Angew. Chem. Int. Ed. Engl. 33:2061, 1994; and Gallop et al., J. Med. Chem. 37:1233, 1994.

Libraries of compounds may be presented in solution (e.g., Houghten, Biotechniques 13:412-421, 1992), or on beads (Lam, Nature 354:82-84, 1991), chips (Fodor, Nature 364:555-556, 1993), bacteria (Ladner, U.S. Pat. No. 5,223,409), spores (Ladner U.S. Pat. No. 5,223,409), plasmids (Cull et al., Proc Natl Acad Sci USA 89:1865-1869, 1992) or on phage (Scott and Smith, Science 249:386-390, 1990; Devlin, Science 249:404-406, 1990; Cwirla et al. Proc. Natl. Acad. Sci. 87:6378-6382, 1990; Felici, J. Mol. Biol. 222:301-310, 1991; Ladner supra.).

In addition, those skilled in the art of drug discovery and development readily understand that methods for dereplication (e.g., taxonomic dereplication, biological dereplication, and chemical dereplication, or any combination thereof) or the elimination of replicates or repeats of materials already known for their anti-inflammatory or anti-neoplastic activity should be employed whenever possible.

Those skilled in the field of drug discovery and development will understand that the precise source of a compound or test extract is not critical to the screening procedure(s) of the invention. Accordingly, virtually any number of chemical extracts or compounds can be screened using the methods described herein. Examples of such extracts or compounds include, but are not limited to, plant-, fungal-, prokaryotic- or animal-based extracts, fermentation broths, and synthetic compounds, as well as modification of existing compounds.

When a crude extract is found to reduce the biological activity of a polypeptide of interest (e.g., BFT-1, BFT-2, BFT-3, STAT-3, IL-17, IL-23), variant, or fragment thereof, further fractionation of the positive lead extract is necessary to isolate chemical constituents responsible for the observed effect. Thus, the goal of the extraction, fractionation, and purification process is the careful characterization and identification of a chemical entity within the crude extract having anti-bacterial, anti-proliferative, or anti-neoplastic activity. Methods of fractionation and purification of such heterogenous extracts are known in the art. If desired, compounds shown to be useful agents for the treatment of inflammatory bowel disease and/or colon carcinogenesis are chemically modified according to methods known in the art.

### Methods of Assaying ETBF Biological Activity

Therapeutics and prophylactics useful in the methods of the invention include, but are not limited to, those that reduce BFT toxicity, those that reduce the survival or proliferation of enterotoxigenic B. fragilis, and/or those that reduce the biological activity or expression of a polypeptide of the invention (e.g., BFT-1, BFT-2, BFT-3, STAT-3, IL-17, IL-23). Neoplastic cell growth is not subject to the same regulatory mechanisms that govern the growth or proliferation of normal cells. Compounds that reduce the growth or proliferation of a hyperplastic or cancerous colon cell are useful for the treatment of neoplasms. Methods of assaying cell growth and proliferation are known in the art. See, for example, Kittler et al. (Nature. 432 (7020):1036-40, 2004) and by Miyamoto et al. (Nature 416(6883):865-9, 2002). Assays for cell proliferation generally involve the measurement of DNA synthesis during cell replication. In one embodiment, DNA synthesis is detected using labeled DNA precursors, such as ([³H]-Thymidine or 5-bromo-2′-deoxyuridine [BrdU], which are added to cells (or animals) and then the incorporation of these precursors into genomic DNA during the S phase of the cell cycle (replication) is detected (Ruefli-Brasse et al., Science 302(5650):1581-4, 2003; Gu et al., Science 302 (5644):445-9, 2003).

Candidate compounds that reduce the survival of a hyperplastic or cancerous cell are also useful as anti-neoplasm therapeutics. Assays for measuring cell viability are known in the art, and are described, for example, by Crouch et al. (J. Immunol. Meth. 160, 81-8); Kangas et al. (Med. Biol. 62, 338-43, 1984); Lundin et al., (Meth. Enzymol. 133, 27-42, 1986); Petty et al. (Comparison of J. Biolum. Chemilum. 10, 29-34, 1995); and Free et al. (AntiCancer Drugs 6: 398-404, 1995). Cell viability can be assayed using a variety of methods, including MTT (3-(4,5-dimethylthiazolyl)-2,5-diphenyltetrazolium bromide) (Barltrop, Bioorg. & Med. Chem. Lett. 1: 611, 1991; Cory et al., Cancer Comm. 3, 207-12, 1991; Paull J. Heterocyclic Chem. 25, 911, 1988). Assays for cell viability are also available commercially. These assays include CELLTITER-GLO® Luminescent Cell Viability Assay (Promega), which uses luciferase technology to detect ATP and quantify the health or number of cells in culture, and the CellTiter-Glo® Luminescent Cell Viability Assay, which is a lactate dehyrodgenase (LDH) cytotoxicity assay.

Candidate compounds that reduce the survival of a hyperplastic or neoplastic cell (e.g., increase cell death, increase apoptosis) are also useful as chemotherapeutics. Assays for measuring cell apoptosis are known to the skilled artisan. Apoptotic cells are characterized by characteristic morphological changes, including chromatin condensation, cell shrinkage and membrane blebbing, which can be clearly observed using light microscopy. The biochemical features of apoptosis include DNA fragmentation, protein cleavage at specific locations, increased mitochondrial membrane permeability, and the appearance of phosphatidylserine on the cell membrane surface. Assays for apoptosis are known in the art. Exemplary assays include TUNEL (Terminal deoxynucleotidyl Transferase Biotin-dUTP Nick End Labeling) assays, caspase activity (specifically caspase-3) assays, and assays for fas-ligand and annexin V. Commercially available products for detecting apoptosis include, for example, Apo-ONE® Homogeneous Caspase-3/7 Assay, FragEL TUNEL kit (ONCOGENE RESEARCH PRODUCTS, San Diego, Calif.), the ApoBrdU DNA Fragmentation Assay (BIOVISION, Mountain View, Calif.), and the Quick Apoptotic DNA Ladder Detection Kit (BIOVISION, Mountain View, Calif.).

### Pharmaceutical Compositions

The present invention contemplates pharmaceutical preparations comprising agents that reduce the expression or biological activity of a polypeptide of interest (e.g., BFT-1, BFT-2, BFT-3, STAT-3, IL-17, IL-23). In one embodiment, the invention provides an effective amount of a STAT-3 inhibitor for use in treating or preventing ETBF-induced colitis, colonic hyperplasia and tumor formation, together with a pharmaceutically acceptable carrier. Agents of the invention may be administered as part of a pharmaceutical composition. The compositions should be sterile and contain a therapeutically effective amount of the agent in a unit of weight or volume suitable for administration to a subject.

These compositions ordinarily will be stored in unit or multi-dose containers, for example, sealed ampoules or vials, as an aqueous solution or as a lyophilized formulation for reconstitution. As an example of a lyophilized formulation, 10 mL vials are filled with 5 mL of sterile-filtered 1% (w/v) aqueous agent solution, such as an aqueous solution of STAT-3 inhibitor, and the resulting mixture can then be lyophilized. The infusion solution can be prepared by reconstituting the lyophilized material using sterile Water-for-Injection (WFI).

The agent may be combined, optionally, with a pharmaceutically acceptable excipient. The term “pharmaceutically-acceptable excipient” as used herein means one or more compatible solid or liquid filler, diluents or encapsulating substances that are suitable for administration into a human. The term “carrier” denotes an organic or inorganic ingredient, natural or synthetic, with which the active ingredient is combined to facilitate administration. The components of the pharmaceutical compositions also are capable of being co-mingled with the molecules of the present invention, and with each other, in a manner such that there is no interaction that would substantially impair the desired pharmaceutical efficacy.

The compositions can be administered in effective amounts. The effective amount will depend upon the mode of administration, the particular condition being treated and the desired outcome. It may also depend upon the stage of the condition, the age and physical condition of the subject, the nature of concurrent therapy, if any, and like factors well known to the medical practitioner. For therapeutic applications, it is that amount sufficient to achieve a medically desirable result.

With respect to a subject having ETBF-induced colitis, colonic hyperplasia and tumor formation, an effective amount is sufficient to stabilize, slow, or reduce inflammation and/or the proliferation of the cancer. Generally, doses of active polynucleotide compositions of the present invention would be from about 0.01 mg/kg per day to about 1000 mg/kg per day. It is expected that doses ranging from about 50 to about 2000 mg/kg will be suitable. Lower doses will result from certain forms of administration, such as intravenous administration. In the event that a response in a subject is insufficient at the initial doses applied, higher doses (or effectively higher doses by a different, more localized delivery route) may be employed to the extent that patient tolerance permits. Multiple doses per day are contemplated to achieve appropriate systemic levels of the compositions of the present invention.

A variety of administration routes are available. The methods of the invention, generally speaking, may be practiced using any mode of administration that is medically acceptable, meaning any mode that produces effective levels of the active compounds without causing clinically unacceptable adverse effects. Modes of administration include oral, rectal, topical, buccal, intracisternal, transdermal, or parenteral routes.

Combination Therapies for the Treatment of Inflammatory Bowel Disease and/or Colon Carcinogenesis

Compositions and methods of the invention may be used in combination with any conventional therapy known in the art. In one embodiment, a composition of the invention having anti-neoplastic activity may be used in combination with an antibiotic therapy known in the art (e.g., metronizole, doxycycline) to reduce or inhibit the proliferation or survival of B. fragilis. In other embodiments, the invention provides for the treatment of ETBF-induced colitis, colonic hyperplasia and tumor formation with a STAT-3 inhibitor to be administered with an antibiotic that reduces or inhibits the survival or proliferation of B. fragilis. In other embodiments, a conventional treatment of colon carcinogenesis is administered in combination with such antibiotics. Convention anti-cancer therapies include, for example, chemotherapy, cryotherapy, hormone therapy, radiotherapy, and surgery. A composition of the invention may, if desired, include one or more chemotherapeutics typically used in the treatment of a colon cancer, such as Folfox Folfury, Erbitox and Avastin. Other examples of chemotherapeutic agents can be found in Cancer Principles and Practice of Oncology by V. T. Devita and S. Hellman (editors), 6th edition (Feb. 15, 2001), Lippincott Williams & Wilkins Publishers.

### Kits

In one embodiment, the invention provides kits for the diagnosis or monitoring of inflammatory bowel disease and/or colon carcinogenesis in a biological sample obtained from a subject. In one embodiment, the kit detects the presence of an ETBF nucleic acid molecule or polypeptide in a biological sample (e.g., stool, urine, blood, serum, tissue). In another embodiment, the kit detects an increase in the level of an ETBF nucleic acid molecule or polypeptide derived from a subject relative to a reference level (e.g., the level present in a control sample obtained from a healthy subject or from the same subject at an earlier time point). In related embodiments, the kit includes reagents for monitoring the expression of an ETBF nucleic acid molecule, such as primers or probes that hybridize to an ETBF nucleic acid molecule. In other embodiments, the kit includes an antibody that binds to an ETBF polypeptide, such as BFT-1, BFT-2 or BFT-3, as well as other isoforms. In other embodiments, the kit comprises a ETBF polypeptide, such as BFT-1, BFT-2 or BFT-3, as well as other isoforms, bound to a substrate. Such a kit is useful for detecting an antibody in a biological sample from a subject to identify a subject that has generated an immune response against ETBF. In one embodiment, the antibody is detected using an ELISA.

In another embodiment, the kit provides immunogenic compositions (e.g., ETBF polypeptide, polynucleotides, killed or attenuated ETBF cells) and methods for the treatment or prevention of ETBF-induced colitis, colonic hyperplasia and tumor formation. In one embodiment, the immunogenic composition is formulated as a vaccine.

Optionally, the kit includes directions for monitoring ETBF nucleic acid molecule or polypeptide levels in a biological sample derived from a subject. In other embodiments, the kit comprises a sterile container which contains the primer, probe, antibody, or other detection regents; such containers can be boxes, ampoules, bottles, vials, tubes, bags, pouches, blister-packs, or other suitable container form known in the art. Such containers can be made of plastic, glass, laminated paper, metal foil, or other materials suitable for holding nucleic acids. The instructions will generally include information about the use of the primers or probes described herein and their use in diagnosing inflammatory bowel disease and/or colon carcinogenesis. Preferably, the kit further comprises any one or more of the reagents described in the diagnostic assays described herein. In other embodiments, the instructions include at least one of the following: description of the primer or probe; methods for using the enclosed materials for the diagnosis of inflammatory bowel disease and/or colon carcinogenesis; precautions; warnings; indications; clinical or research studies; and/or references. The instructions may be printed directly on the container (when present), or as a label applied to the container, or as a separate sheet, pamphlet, card, or folder supplied in or with the container.

The practice of the present invention employs, unless otherwise indicated, conventional techniques of molecular biology (including recombinant techniques), microbiology, cell biology, biochemistry and immunology, which are well within the purview of the skilled artisan. Such techniques are explained fully in the literature, such as, “Molecular Cloning: A Laboratory Manual”, second edition (Sambrook, 1989); “Oligonucleotide Synthesis” (Gait, 1984); “Animal Cell Culture” (Freshney, 1987); “Methods in Enzymology” “Handbook of Experimental Immunology” (Weir, 1996); “Gene Transfer Vectors for Mammalian Cells” (Miller and Calos, 1987); “Current Protocols in Molecular Biology” (Ausubel, 1987); “PCR: The Polymerase Chain Reaction”, (Mullis, 1994); “Current Protocols in Immunology” (Coligan, 1991). These techniques are applicable to the production of the polynucleotides and polypeptides of the invention, and, as such, may be considered in making and practicing the invention. Particularly useful techniques for particular embodiments will be discussed in the sections that follow.

The following examples are put forth so as to provide those of ordinary skill in the art with a complete disclosure and description of how to make and use the assay, screening, and therapeutic methods of the invention, and are not intended to limit the scope of what the inventors regard as their invention.

### EXAMPLES

### Example 1

### ETBF Stimulates Rapid Colitis and Colon Tumors in Min Mice

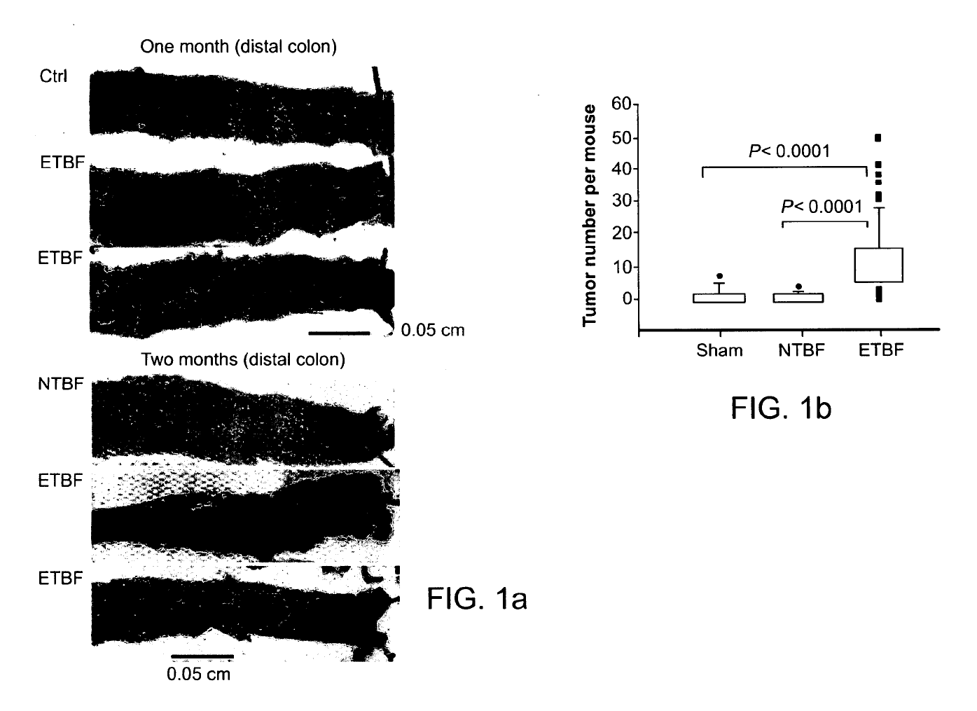

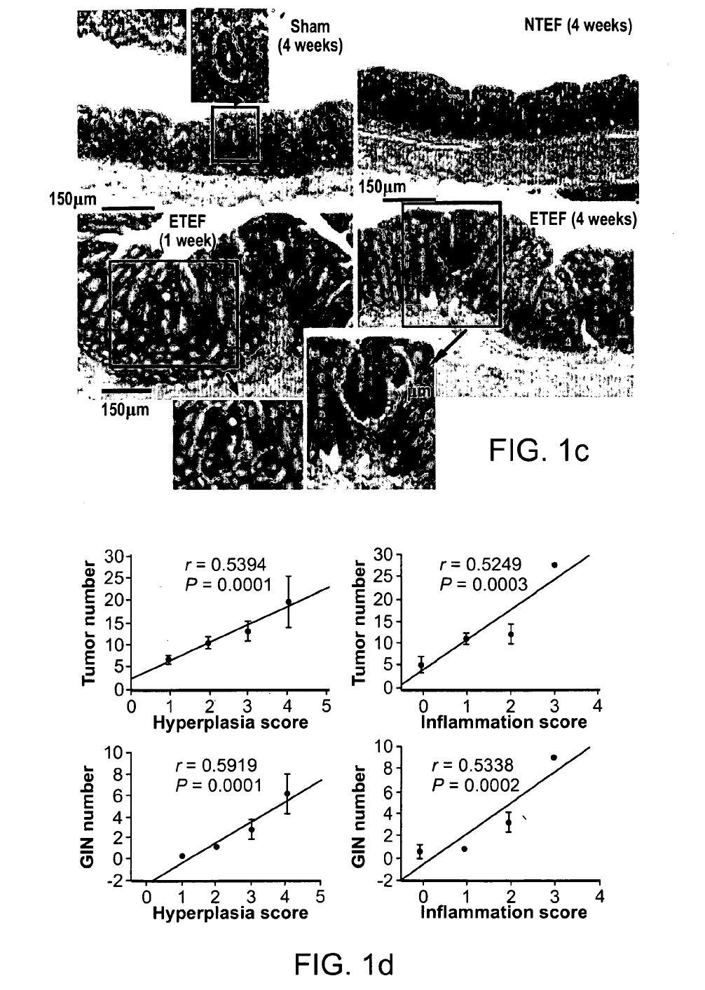

Min mice colonized with enterotoxigenic Bacteroides fragilis, but not nontoxigenic B. fragilis (NTBF), usually developed brief diarrhea by 2-3 days, with resolution of the symptoms 4-5 days after colonization. Asymptomatic high-level colonization (≧1×10⁹ colony-forming units per g feces) with NTBF or ETBF occurred by day 3 after infection and persisted. Only ETBF-colonized mice showed a marked increase in colonic thickness, inflammation and visible colonic tumors, especially distally, at 4 weeks or later (FIG. 1a-c and Table 1).

TABLE 1
Min mouse colon histological scores 1 week and 4-6 weeks
after ETBF or NTBF colonization

| | Median (range) | | | |
|---|---|---|---|---|
| | Inflammation | Hyperplasia | GIN | Gross tumors |
| 1 week | | | | |
| Sham (n = 6) | 0 (0-0) | 1 (0-1) | 0 (0-0) | NA |
| NTBF (n = 4) | 0 (0-0) | 0 (0-0) | 0 (0-0) | NA |
| ETBF (n = 16) | 2 (0-3)^{a} | 3 (2-4)^{b} | 1.5 (0-4)^{c} | NA |
| 4-6 weeks | | | | |
| Sham (n = 9) | 0 (0-1) | 0 (0-1) | 0 (0-0) | 2 (0-8) |
| NTBF (n = 5) | 0 (0-0) | 0 (0-1) | 0 (0-0) | 3 (2-4) |
| ETBF (n = 59) | 1 (0-3)^{d} | 2 (1-4)^{e} | 1 (0-16)^{f} | 9 (2.49)^{g} |

NA, not applicable.
^{a}P < 0.015 versus 1 week sham and NTBF; independent comparisons.
^{b}P < 0.0004 versus 1 week sham and NTBF; independent comparisons.
^{c}P < 0.042 versus 1 week sham and NTBF; Independent comparisons.
^{d}P < 0.0006 versus 4-6 week sham and NTBF; independent comparisons.
^{e}P < 0.0004 versus 4-6 week sham and NTBF; independent comparisons.
^{f}P < 0.018 versus 4-6 week sham and P = 0.10 versus NTBF; independent comparisons.
^{g}P < 0.0005 versus 4-6 week sham and NTBF; independent comparisons.

Histopathology of ETBF-colonized colons confirmed increases in inflammation, hyperplasia and gastrointestinal intraepithelial neoplasia (GIN) foci compared to sham-treated or NTBF-infected colons (Table 1 and FIG. 1c). Linear regression analysis of inflammation or hyperplasia severity supported an association between ETBF-induced inflammation or hyperplasia with GIN and gross colon tumor detection (FIG. 1d). Furthermore, GIN, inflammation and hyperplasia were detected only in ETBF-colonized colons at 1 week after colonization (FIG. 1c). These data suggest that ETBF induces de novo tumor formation quickly and may enhance tumor growth rates. Tumors in ETBF-colonized mice were typically laden with inflammatory infiltrates comprised of granulocytes and mononuclear cells not seen in tumors in sham-inoculated or NTBF-colonized Min mice (FIG. 1c). No increase in the number of small bowel tumors was observed between experimental groups, consistent with the known colonic niche for B. fragilis colonization.

### Example 2

### ETBF Selectively Activates Stat3 in the Colon

To address the mechanisms of ETBF-induced colitis and carcinogenesis, the activation of Stat proteins was assessed. Stat proteins are a family of transcription factors activated by cytokine receptor signaling through tyrosine phosphorylation with nuclear translocation and are central to the regulation of immune responses. Stat1 and Stat4 contribute to TH1-dependent immune responses, whereas Stat6 has a key role in TH2 responses. Stat3 transduces signals from numerous growth factor and cytokine receptors, is constitutively activated in diverse cancers and is absolutely required for TH17 cell generation while simultaneously negatively regulating TH1-mediated inflammation.

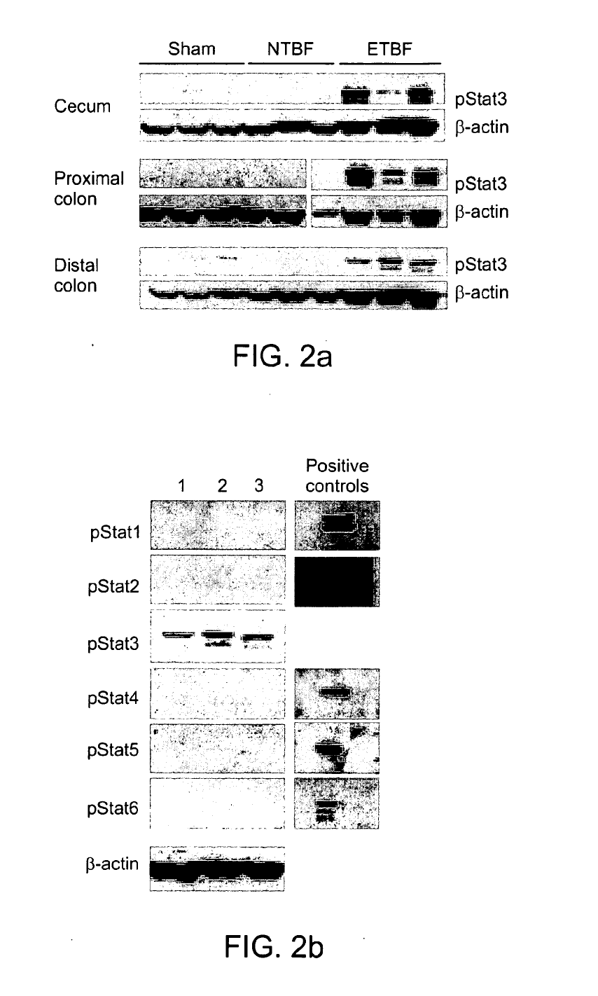

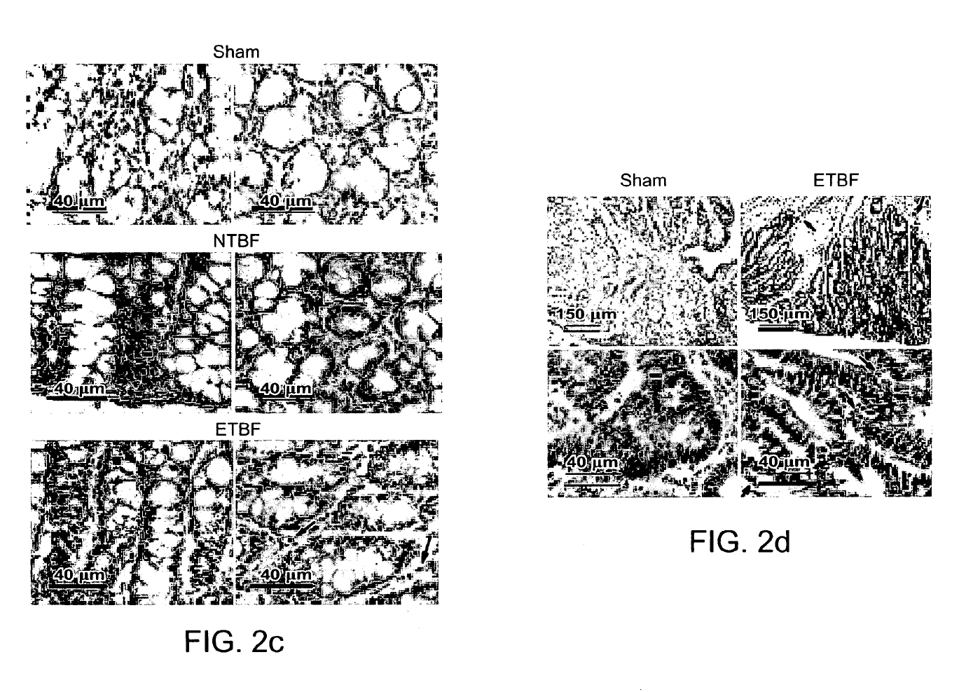

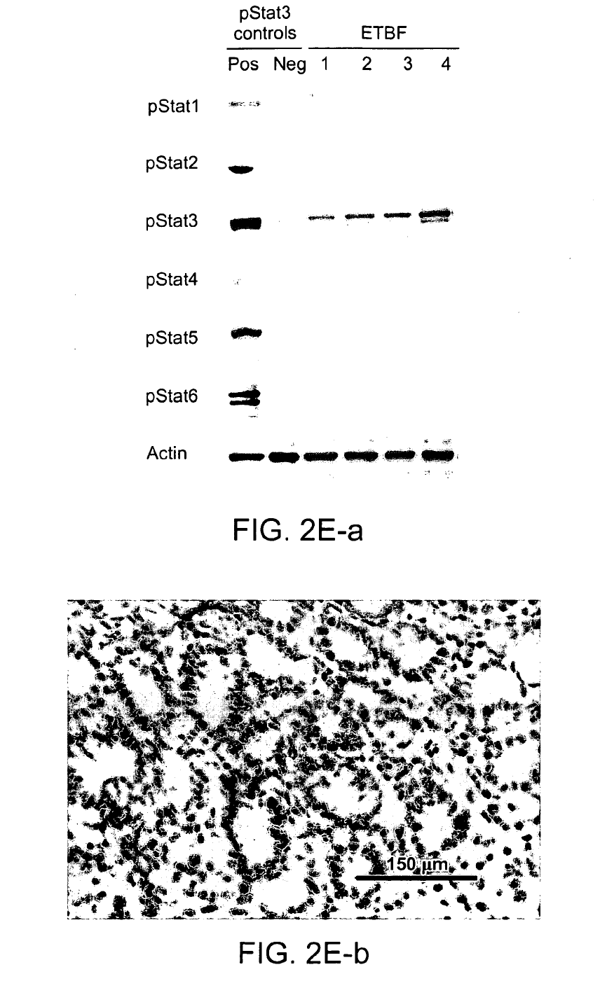

Using antibodies specific for each phosphorylated Stat protein, only phosphorylated Stat3 (pStat3) was found to be abundant in the colonic mucosa of ETBF-colonized Min mice at 2 days after infection (FIG. 2a), whereas only faint pStat3 staining was observed in some sham or NTBF-colonized Min mouse colons (FIG. 2a). pStat1, pStat2, pStat5 or pStat6 were not detected in the colons of any mouse experimental group (FIG. 2b). Very faint pStat4 signals were detected in some ETBF-colonized Min mice. The colons of wild-type (WT) C57BL/6 mice revealed identical pStat staining (FIG. 2E-a), indicating that the highly selective activation of predominantly Stat3 by ETBF colonization is independent of the Apc mutation in Min mice.

Immunohistochemistry was used to examine the cellular localization and time course of pStat3 activation in ETBF-colonized mice. Stat3 activation occurred in colonic epithelial cells and a subset of infiltrating immune cells in ETBF-colonized Min mice at 2 days to 4 weeks after colonization compared to pStat3 staining in NTBF-colonized or sham mice at the same time points (FIG. 2c and FIG. 2E-b). In addition to nontumorous epithelium, 13 tumors of variable sizes were found in random colon histopathology sections of seven ETBF-colonized Min mice, and all 13 tumors showed intense epithelial cell pStat3 activation and pStat3 staining in a subset of mucosal immune cells (FIG. 2d). The low frequency of tumors in NTBF-colonized or sham-treated Min mice limited detection of tumors in random colon sections. However, in three tumors that were identified in NTBF-colonized or sham Min mice sections, the pStat3 staining was less consistent and less intense, particularly in the epithelial compartment (FIG. 2d). Thus, beyond inducing tumors in Min colons, ETBF colonization quantitatively altered at least one oncogenic signaling pathway in already established tumors.

### Example 3

### ETBF Induced Dominant Colonic Th17 Inflammatory Infiltrates

Stat3 signaling functions in the generation of TH17 cells, and pStat3 binds the Il17a and Il17f promoters. To determine whether pStat3 activation by ETBF colonization of Min mice initiates a TH17 mucosal immune response, FACS analysis (n=8 experiments) of isolated intraepithelial lymphocyte and lamina propria lymphocyte populations was used. This analysis showed an approximately four- to fivefold higher number of CD4⁺ T cells in the lamina propria of ETBF-colonized Min mice after 1 week as compared to NTBF-colonized or sham Min mice.

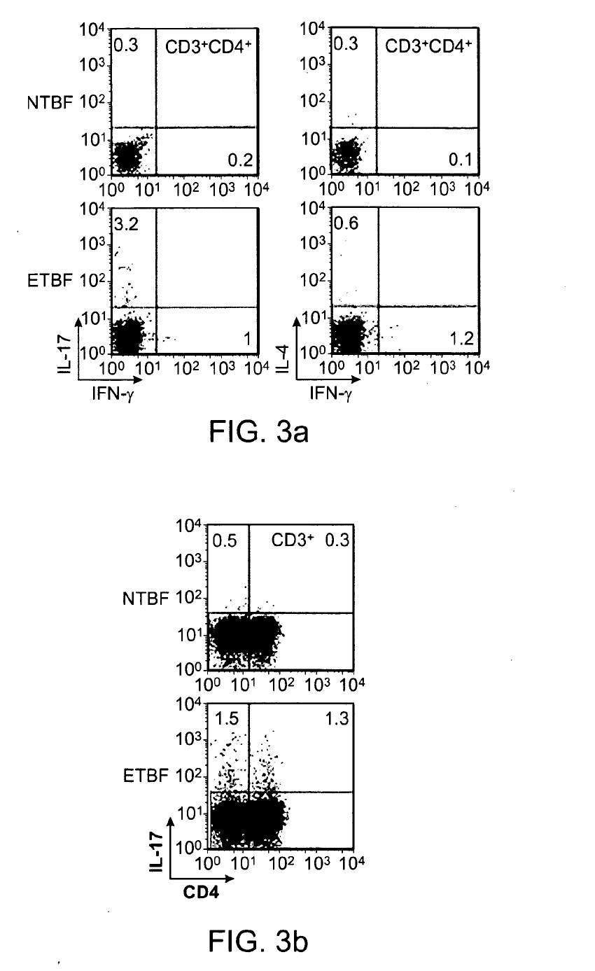

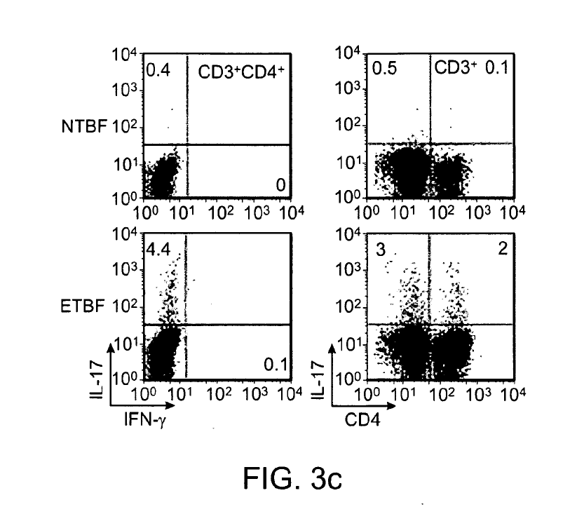

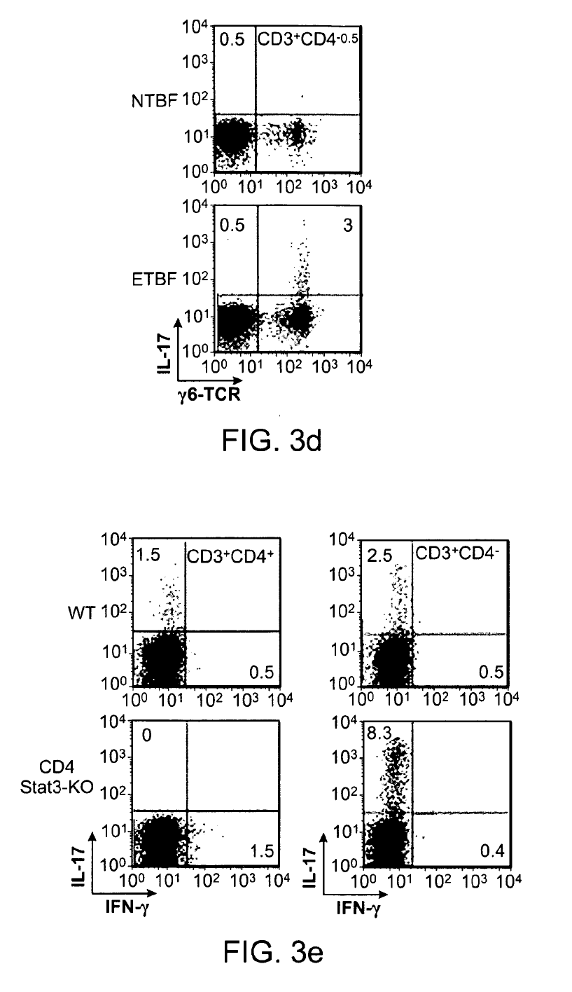

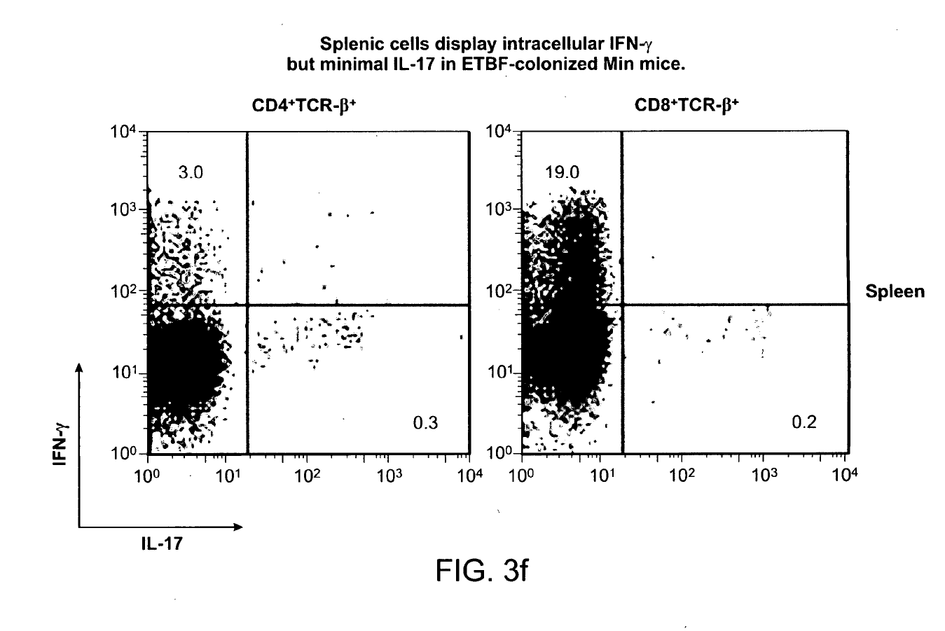

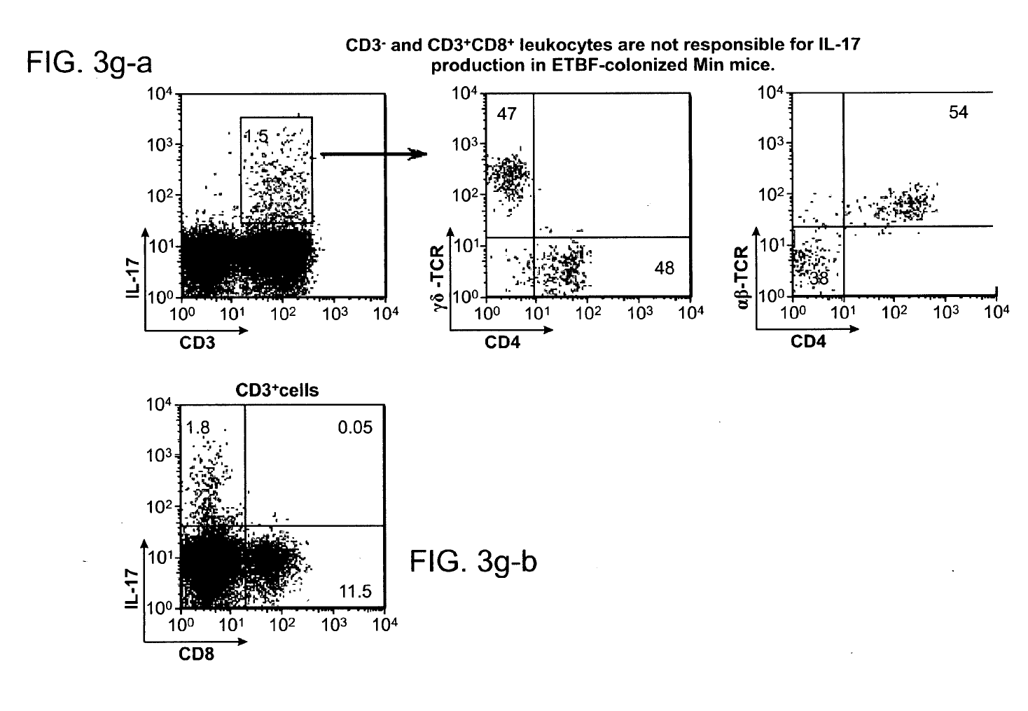

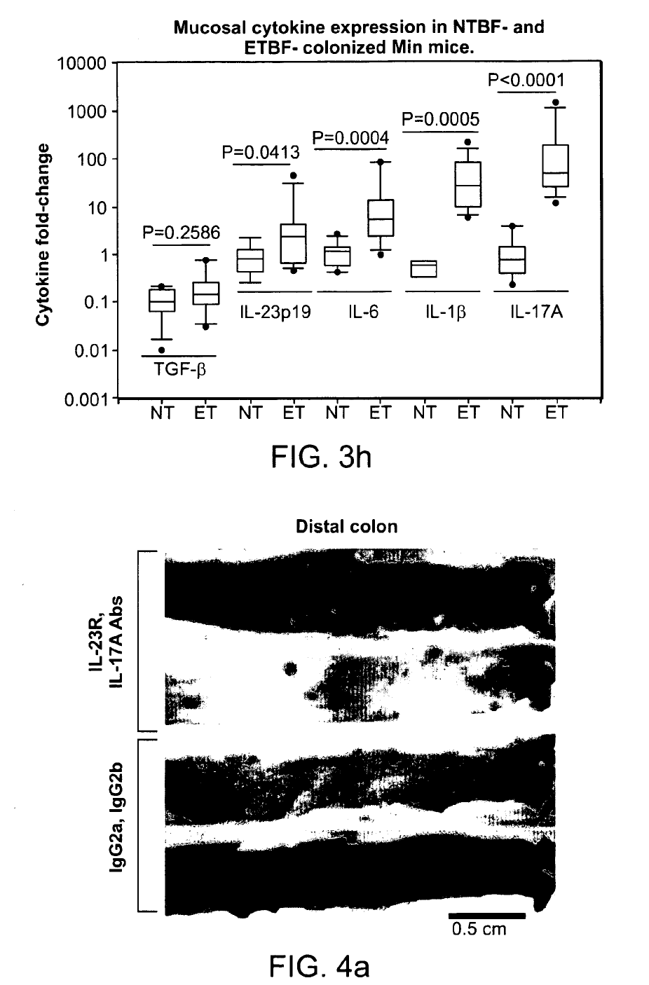

ETBF-colonized Min mice indeed developed a strongly skewed TH17 response characterized by equally contributory IL-17-secreting CD3⁺CD4⁺ and CD3⁺CD4⁺ effector populations in the lamina propria (FIG. 3a,b). No expanded IL-4-producing T cell effector populations were found, and the modest number of IFN-γ-producing CD3⁺CD4⁺ T cells produced low amounts of IFN-γ (FIG. 3a). In contrast to colonic lymphocytes, CD4⁺TCRαβ⁺ and CD8⁺TCRαβ⁺ splenic cells isolated from ETBF-colonized mice showed enhanced IFN-γ staining with minimal IL-17 production (FIG. 3F). Similar results were obtained for WT mice (FIG. 3c).

To further identify the IL-17-producing mucosal T cell populations in ETBF-colonized Min mice, antibodies were used to distinguish between classical (TCRαβ⁺-bearing) and nonclassical (TCRγδ-bearing) T cells. IL-17 production by CD3⁺CD4⁻ T cells in ETBF-colonized Min or WT mice was attributable to CD3⁺TCRγδ⁺ lamina propria cells (FIG. 3d). In contrast, neither lamina propria CD3⁺CD8⁺ cells nor CD3⁻ cells showed intracellular IL-17 staining in ETBF-colonized Min or WT mice (FIG. 3G-a and 3G-b).

Beyond Stat3 activation, induction of a TH17 immune response typically requires IL-6, which, together with transforming growth factor-β(TGF-β) (and augmented by IL-1β), induces TH17 differentiation, whereas expansion of IL-17-producing CD4+ lymphocytes is promoted by IL-23. Thus, to determine whether Stat3 is required for ETBF-induced IL-17 production by colonic CD3⁺CD4⁺ T cells isolated from ETBF-colonized WT mice with functional Stat3 knockout in the CD4 T cell compartment (CD4 Stat3-KO). CD4-targeted Stat3 knockout obliterated ETBF induction of IL-17 in this T cell subset, whereas IL-17 persisted in Stat3-competent CD3⁺CD4⁻ T cells (FIG. 3e). Histopathology of ETBF-colonized, CD4-targeted, Stat3-knockout mice revealed significant decreases in inflammation and hyperplasia compared to littermate Stat3-sufficient mice, consistent with the contribution of CD4+ cells and Stat3 signaling to ETBF colitis (P≦0.03, Table 2).

TABLE 2
Mouse colon histology scores 1 wk after NTBF or ETBF
colonization in wild-type (WT) or CD4 Stat3-KO C57BI/6 mice.

Median (range)

| | Inflammation | Hyperplasia |
|---|---|---|
| Sham WT (n = 3) | 0 (0-0) | 0 (0-0) |
| Sham CD4 Stat3-KO (n = 4) | 0 (0-1) | 0.5 (0-1) |
| NTBF WT (n = 4) | 0 (0-0) | 0.5 (0-1) |
| NTBF CD4 Stat3-KO (n = 4) | 0 (0-1) | 0.5 (0-1) |
| ETBF WT (n = 13) | 3 (1-4)^{a,b} | 2 (2-3)^{a,d} |
| ETBF CD4 Stat3-KO (n = 18) | 2 (1-3)^{c} | 2 (1-3)^{e} |

^{a}P ≦ 0.004 vs sham and NTBF WT, independently
^{b}P ≦ 0.001 vs ETBF CD4 Stat3-KO
^{c}P ≦ 0.005 vs sham and NTBF CD4 Stat3-KO, independently
^{d}P ≦ 0.03 vs ETBF CD4 Stat3-KO
^{e}P ≦ 0.009 vs sham and NTBF CD4 Stat3-KO, independently

By quantitative RT-PCR (qRT-PCR), markedly higher levels of IL-17 messenger RNA were detected in the colonic mucosa of ETBF-colonized Min and WT mice relative to NTBF-colonized mice at 1 week after colonization (FIG. 3H), consistent with the detection of IL-17 protein in CD4⁺ and CD4⁻ T lymphocytes (FIG. 3b). Higher levels of IL-1β, IL-6, IL-23 and TGF-β mRNA were also found 1 week after ETBF colonization, although the differences in TGF-β mRNA were not significant (FIG. 3H).

A major component of TGF-β regulation occurs after transcription; thus, its mRNA levels were less informative than the mRNA levels of the other cytokines. Lastly, sorted CD3⁺CD4⁺ lymphocytes from Min mice colonized with ETBF or NTBF were examined by qRT-PCR for induction of the gene encoding the TH17-specific transcription factor, RORγt23, in parallel with the Il17a gene. In CD3⁺CD4⁺ T cells isolated from the colons of ETBF-colonized mice, expression of the gene encoding RORγt was tenfold higher (±2.6, mean±s.e.m.) and Il17a gene expression was 21-fold higher (±2.6) compared to NTBF-colonized mice.

### Example 4

### Blockade of IL-17 Inhibits ETBF-Induced Colon Tumors

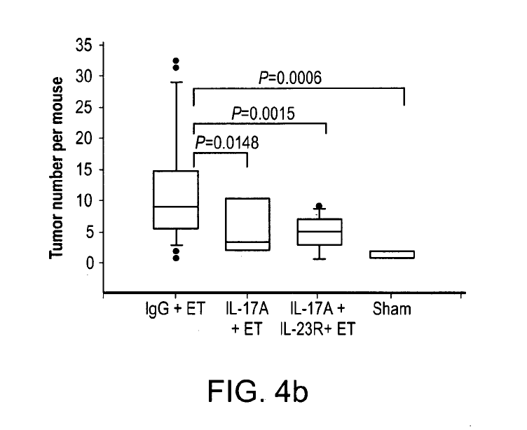

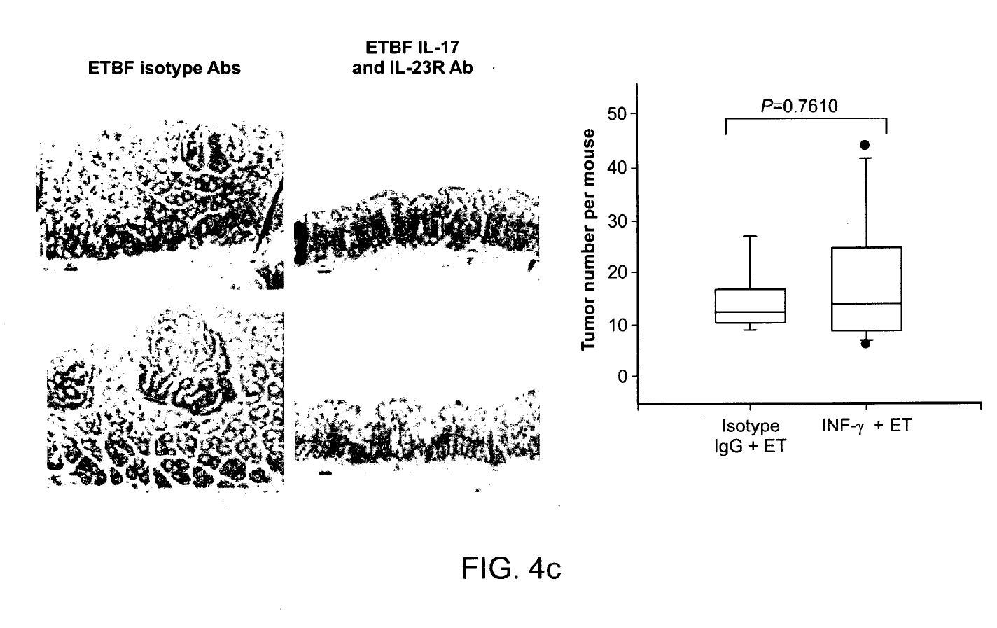

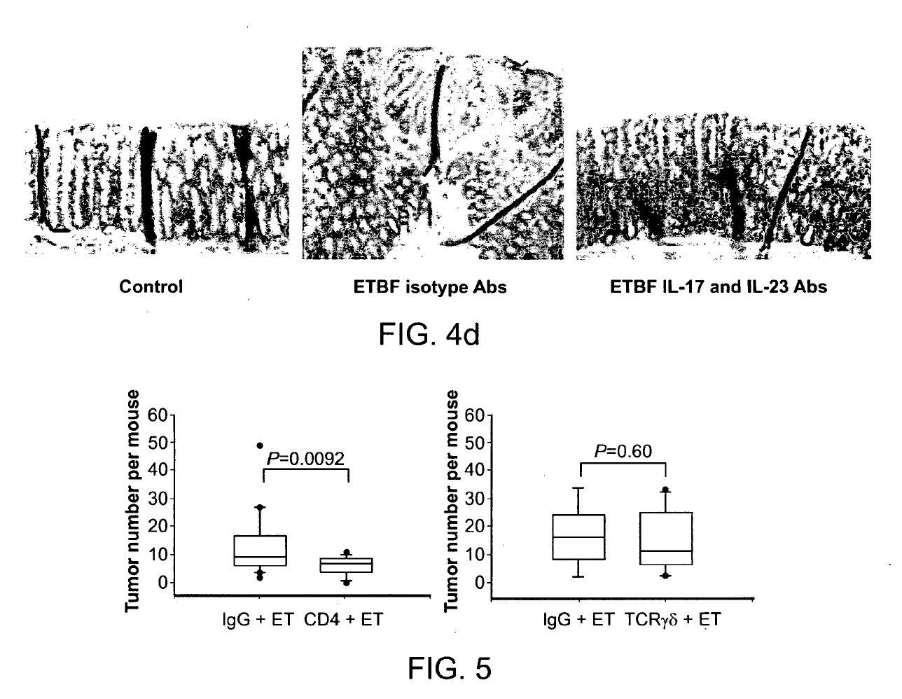

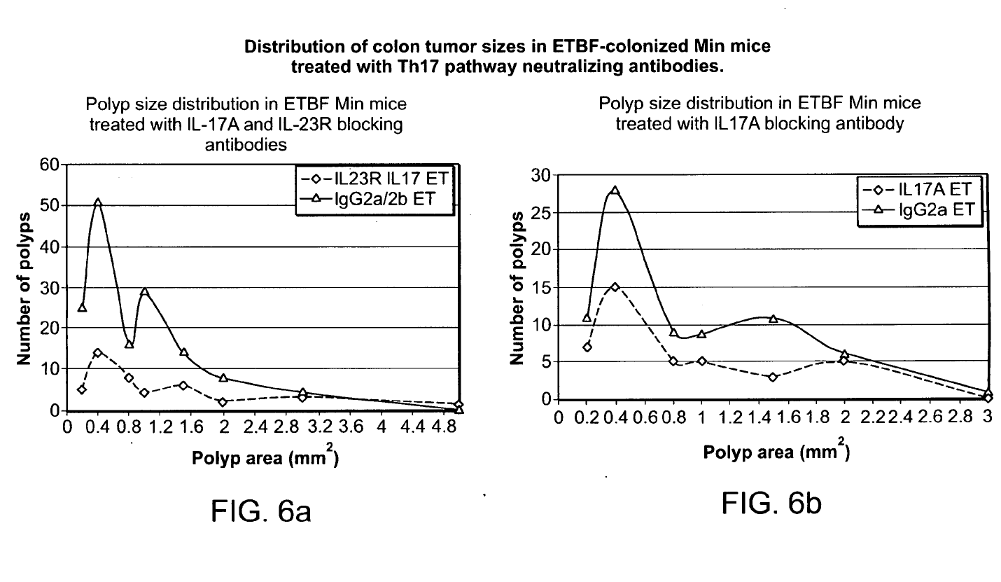

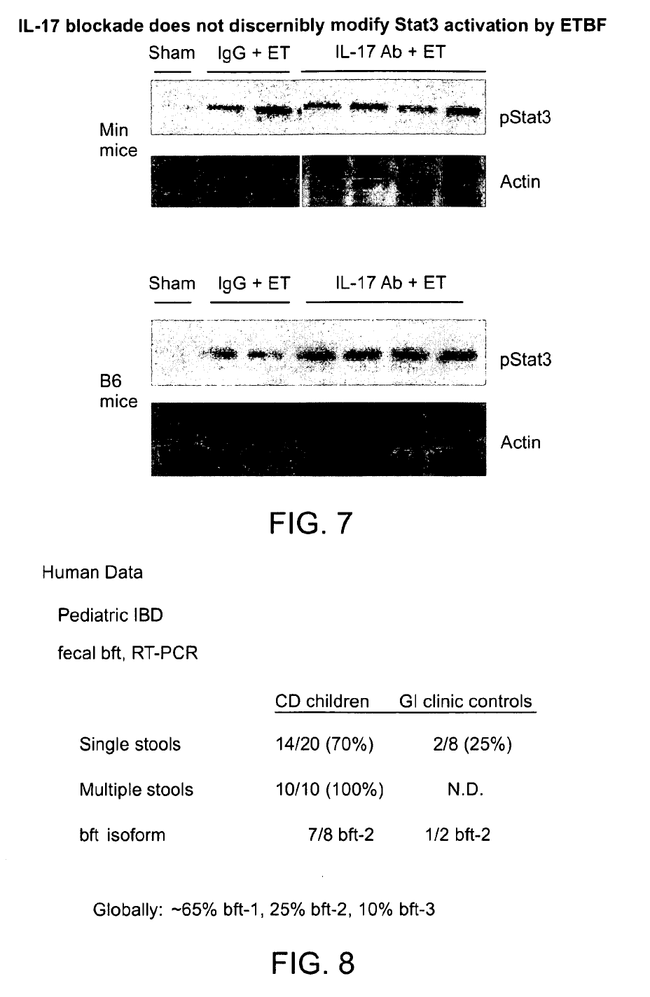

To evaluate the contribution of TH17 inflammatory cells to ETBF-induced tumor formation in Min mice, experiments were conducted with IL-17A-, IL-23 receptor (IL-23R)— or IFN-γ-neutralizing antibodies. Of the six isoforms of IL-17, IL-17A predominates in humans and mice and in the colonic mucosa after 1 week in ETBF-colonized Min or WT mice (FIG. 3H). Blockade of IL-17A alone or combined blockade with IL-23R significantly inhibited colon tumor formation at 5 weeks after colonization (FIG. 4a,b). The size distribution of the tumors did not differ between the mice treated with IL-17- and IL-23R-neutralizing antibodies mice compared to isotype controls (FIG. 6A, 6B), emphasizing the contribution of the TH17 response in tumor initiation and suggesting a minor role in tumor growth rate. In contrast, IFN-γ blockade did not modify ETBF-induced colon tumorigenesis (FIG. 4b). IL-17A blockade did not detectibly modify pStat3 levels, as determined by western blotting, nor did it affect the cellular distribution of pStat3, as determined by immunohistochemistry, in the colons of ETBF-colonized WT or Min mice, suggesting that Stat3 activation is upstream of IL-17 induction (FIG. 6A, 6B).

Histopathology revealed marked inhibition of colonic mucosal proliferation with fewer infiltrating leukocytes (FIG. 4c) and GIN foci on random colon tissue sections in ETBF-colonized Min mice treated with IL-17A-blocking antibodies or both IL-17A- and IL-23R-blocking antibodies for 5 weeks compared to ETBF-colonized mice treated with isotype control antibodies for 5 weeks (P<0.02, Table 3).

TABLE 3
Min mouse colon histology scores 4-5 wks after ETBF
colonization in mice treated with isotype antibodies or
IL-17 and IL-23R neutralizing antibodies or IL-17
neutralizing antibodies alone

| | Median (range) | | | |
|---|---|---|---|---|
| | Inflam- | Hyper- | | Gross |
| | mation | plasia | GIN | Tumors |
| ETBF (isotype Abs) | 2 (1-3) | 2 (2-3) | 2 (0-6) | 10 (2-32) |
| (n = 24) | | | | |
| ETBF | 1 (0-1)^{a} | 2 (1-2)^{b} | 0 (0-1)^{b} | 5 (1-9)^{c} |
| (IL-17/IL-23R Abs) | | | | |
| (n = 14) | | | | |
| ETBF (IL-17 Abs) | 1 (1-1)^{d} | 1 (0-2)^{b} | 0.5 (0-2)^{e} | 3.5 (1-13)^{e} |
| (n = 8) | | | | |

All comparisons are to ETBF (isotype antibodies).
^{a}P < 0.004
^{b}P < 0.0007
^{c}P < 0.002
^{d}P = 0.043
^{e}P < 0.02

Similar results were obtained in ETBF-colonized Min mice treated with IL-17A and IL-23R blockade for 1 week (FIG. 4d).

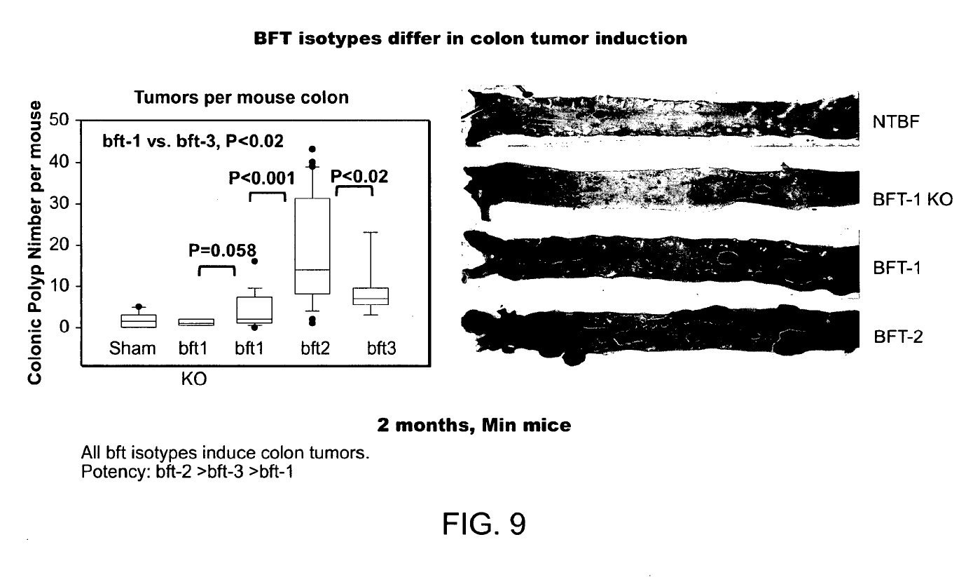

The question of whether depletion of CD4⁺ or TCRγδ⁺ T lymphocytes modifies tumor induction in ETBF-colonized Min mice was addressed. Antibody-mediated depletion of CD4⁺T lymphocytes significantly inhibited the accelerated tumor formation detected 4-6 weeks after ETBF inoculation when compared to ETBF-colonized mice treated with an isotype control antibody (P=0.009), whereas TCRγδ⁺ T cell depletion did not modify ETBF-induced colon tumors (FIG. 5). This result provides a direct example that endogenous CD4⁺T responses contribute to infection-induced carcinogenesis. Different BFT isoforms may differ in the tumorogenic ability (FIG. 9).

The results presented herein are the first to demonstrate a direct role for endogenous T cell immune responses in infection-induced carcinogenesis. Intracellular cytokine staining (ICS) and in vivo antibody blockade experiments further indicated a TH17 response, driven by Stat3 activation, as acting in the procarcinogenic effect. This newly described mechanism for infection-induced carcinogenesis may be highly relevant for human carcinogenesis, as, in contrast to all other models of murine colitis, the present model uses colonization with a human commensal bacterium, ETBF. In this model, essentially all of the IL-17 production was similarly distributed between two T cell subsets, CD4⁺TCRαβ⁺ and CD4⁻8⁻TCRαβ⁺ T cells. The in vivo depletion experiments emphasized the contribution of classical CD4⁺TH17 cells in ETBF-induced colon tumorigenesis, as CD4⁺, but not TCRγδ⁺, T cell depletion markedly lowers tumor number. Without wishing to be bound by theory, it is still possible that colonic γδ T cells contributed to the overall tumorigenesis process but that IL-17 production by CD4⁺TH17 cells were sufficient to induce tumorigenesis in the absence of γδ T cells.

In addition to promoting TH17 development and IL-17 transcription, Stat3 activation in the tumor microenvironment inhibits IL-12p35 transcription while enhancing IL-23p19 transcription, thereby shifting the balance from IL-12 to IL-23 (ref. 24). This finding, together with the finding that 9,10-dimethyl-1,2-benzanthracene-induced skin carcinogenesis is diminished in IL-23p19-knockout mice and enhanced in IL-12p35-knockout mice 25 as well as the results presented here, suggest that Stat3 potentially promotes a complex procarcinogenic TH17-type immune response. Beyond the immune compartment, Stat3 activation in the intestinal epithelial compartment also contributes to colon carcinogenesis in the axozymethane with dextran sulfate sodium model (Bollrath et al. Cancer Cell 15, 91-102 (2009); Grivennikov et al. Cancer Cell 15, 103-113 (2009). Tissue-selective Stat3 knockouts on the Min background will be necessary to define the specific roles of Stat3 activation in the various cell types in ETBF colitis.

Although recent work on inflammation-induced carcinogenesis has focused on innate pathways, particularly the NF-κB and myeloid differentiation factor-88 pathways, little is known about the direct role of adaptive responses in general and T cell responses in particular. In a transgenic model of skin carcinogenesis driven by keratinocyte-specific expression of the human papillomavirus-16 E6 and E7 oncogenes, B lymphocytes proved to be major promoters of tumor formation. Recent adoptive transfer studies of activated T cells into Rag2^{-/-} (T and B cell deficient)×Min mice and Ifng^{-/-} TCR transgenic T cells into RIP1-Tag2 (rat insulin promoter driving T antigen expression) transgenic mice that develop islet cell tumors demonstrated the potential for T cells to promote tumor development. However, no previous study has yet documented a direct role for endogenous T cell responses as a mechanism for infection-induced cancer.

In contrast, a number of recent studies in Rag-knockout mice and mice deficient in interferon signaling show a clear role for lymphocytes in inhibiting cancer development and forcing emerging tumors to edit themselves to evade immune elimination. The finding that the procarcinogenic T cell response in our system is TH17 mediated suggested that the role of T cell responses in inhibiting or promoting carcinogenesis may depend on the qualitative response. Stat3-driven TH17 responses, characterized by production of IL-17A in mice and humans and driven by IL-23, are crucial in mucosal inflammatory responses in the lung and gut and are implicated in a number of autoimmune disorders.

Although the mechanisms by which ETBF-induced TH17 responses promote colon carcinogenesis remain undefined, two notable histopathological findings in ETBF-colonized colons reported herein are the marked epithelial hyperproliferative response and the inflammatory infiltrates, both of which were substantially lessened upon in vivo blockade with IL-17- and IL-23R-blocking antibodies. Abundant granulocytes were observed in ETBF-colonized colons, consistent with the reported role of TH17 responses in amplifying granulocytic inflammatory responses. The question of whether IL-17 and other TH17 cytokines promote colonic epithelial hyperproliferation and whether specific TH17-induced granulocyte products such as reactive oxygen or nitrogen species contribute to the rapid GIN induction (1 week) by ETBF is under evaluation.

Although the intestinal TH17 mucosal response to ETBF is not unique, the rapid induction of colonic tumors in young Min mice is unique among reported data on enteric pathogens. Two mouse enteric pathogens, Citrobacter rodentium and Helicobacter hepaticus, may induce colonic mucosal TH17 immune responses. However, IL-23 deficiency results in a fatal colitis in mice infected with C. rodentium, but diminished colitis in mice infected with H. hepaticus, suggesting pathogen-specific roles for TH17 immunity in colitis. Both of these nonhuman enteric pathogens can induce colonic tumors in Min mice, with C. rodentium inducing modest colonic tumor induction after 5 months and H. hepaticus-associated colonic oncogenesis observed only in aged immune-insufficient mice, such as Rag2^{-/-}×Min or 129/SvEv Rag2^{-/-} mice or mice with, for example, defective TGF signaling Salmonella typhimurium induces TH17-associated ileitis in rhesus macaques, and uncharacterized commensal flora in mice induce CD4+ TH17 cells with colitis induction upon adoptive transfer to Rag1^{-/-} mice, but links to colonic tumor pathogenesis have not been reported.

NTBF strains that do not secrete BFT, the only identified ETBF virulence factor, do not stimulate colonic Stat3 activation, TH17 mucosal immune responses nor enhance colonic tumor formation in Min mice, indicating that BFT has a central role in triggering a procarcinogenic colonic mucosal response. Without wishing to be bound by theory, mechanistic data suggest that BFT acts as an oncogenic bacterial toxin through cleavage of E-cadherin, a tumor suppressor protein, triggering β-catenin nuclear signaling and colonic epithelial cell proliferation. BFT also triggers activation of NF-κB, resulting in colonic epithelial cell secretion of proinflammatory cytokines. Without wishing to be bound by theory, the data indicate that ETBF is a human oncogenic bacterium, owing to its production of BFT in vivo and its association with colonic inflammation. Colonic tumor induction by ETBF in human populations would probably require long-term colonization. Although longitudinal carriage of B. fragilis is poorly characterized, ETBF is prevalent, at least in some locales, with 4-35% of studied populations showing asymptomatic fecal carriage.

Commensal colonic bacteria are often cited as crucial environmental factors influencing the development of colorectal cancer, but linkages to specific organisms and the mechanisms promoting oncogenesis have been tenuous. Accordingly, this is the first report of an oncogenic human colonic commensal organism, and the data provided herein are reminiscent of early studies of H. pylori, an ancient gastric commensal, colonizing more than 50% of the global population, that routinely induces gastritis and, infrequently, also induces gastric cancer. The mucosal immune response to H. pylori is also TH17 skewed, consistent with our observations linking ETBF-induced colonic mucosal TH17 inflammation to colonic tumor formation. Together, these observations underpin the necessity of human studies to identify potential links between ETBF colonization, colonic Stat3 activation, colonic TH17 responses and human colorectal cancer.

### Example 5

### B. fragilis Toxin is Associated with Human Pediatric Crohn's Disease

RT-PCR was used to detect expression of the B. fragilis gene that encodes enterotoxin in stool from human subjects diagnosed as having Crohn's disease or from control subjects. The RT-PCR was carried out on DNA purified from the stool using primers flanking the gene. Surprisingly, 70% of pediatric patients diagnosed as having Crohn's disease also showed B. fragilis toxin present in their stool. When multiple stool samples were analyzed, 100% of the pediatric patients diagnosed as having Crohn's disease were positive for B. fragilis toxin (FIG. 8). Further, a preponderance of bft-2 was detected in the fecal specimens compared to the current understanding of the global distribution of bft genes in ETBF strains where bft-1 predominantes (FIG. 8).

### Example 6

### Detection of an ETBF Nucleic Acid Molecule in a Subject Sample

The invention provides methods for detecting ETBF nucleic acid molecules in biological samples (e.g., stool) from a subject. In one embodiment, the nucleic acid molecules are purifed from said sample, amplified using a primer sequence described herein, and the amplicons are detected using any method known in the art. In one example, primers flanking bft-1, -2, or -3 are used. In another example, nested primers are used. In another example, a hybridization probe is used to detect the presence of the amplicon.

| | 5′ Position |
|---|---|
| Primers for Nested PCR: | |
| RS-3: TGA AGT TAG TGC CCA GAT GCA GG | 705 bft |
| RS5-: CA TCT TAT TCC ATT AAT CGA ACT TCG | 48 bp downstream bft |
| Inner primers | |
| RS-1A: TGC GGC GAA CTC GGT TTA TGC | 729 bft |
| RS-2: AGC TGG GTT GTA GAC ATC CCA CTG G | 1019 bft |
| SYBRGreen: | |
| BFT-F-619: 5′ gaaagtcagacacgtgcagtacc | 618 bft |
| BFTR-727: 5′ cctgcatctgggcactaac | 726 bft |
| TaqMan | |
| Primer Forward: TGGCGAATCCATCAGCTACA | 351 bft |
| Primer Reverse: TCGGCAATCTCATTCATCATTT | 410 bft |
| Probe: CGCATACAAGGAAGC | 372 bft |

In another embodiment, any of the following primers are used.

| Name of | |
|---|---|
| primer | Sequence |
| BFT-F-Universal | GAACCTAAAACGGTATATGT |
| BFT-R-Universal | GTTGTAGACATCCCACTGGC |
| BFT-1R2 | TCCCTCTTTGGCGTCGCCA |
| BFT-2R | CGCTCGGGCAACTAT |
| BFT-3R2 | CAAAATGTTGTTGTCCCAAGTT |

| | | |
|---|---|---|
| Bft: | BFT-F-Universal/BFT-R-Universal | Product size = 368 bps |
| Bft-1: | BFT-F-Universal/BFT-1R2 | Product size = 190 bps |
| Bft-2: | BFT-F-Universal/BFT-2R | Product size = 175 bps |
| Bft-3: | BFT-F-Universal/BFT-3R2 | Product size = 287 bps |

In another embodiment, an ETBF nucleic acid molecule is amplified using PCR as follows:

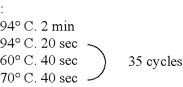

The results reported herein were obtained using the following methods and materials.

### Bacteriology.

ETBF strain 86-5443-2-2 (secretes BFT-2); and NTBF strain9343 (American Type Culture Collection) were grown overnight anaerobically at 37° C.

### Mice.

MinApc716^{+/-} mice (expressing a mutant gene encoding an adenomatous polyposis coli protein truncated at amino acid 716), C57BL/6 mice (either wild-type littermates of MinApc716^{+/-} mice or from Jackson Laboratories) and conditional CD4 Stat3-KO mice (CD4-Cre×Stat3flox/flox mice) on a C57BL/6 background generated as described (Harris, T. J. et al J. Immunol. 179, 4313-4317 (2007)) were specific pathogen free. To enhance B. fragilis colonization, clindamycin (0.1 g 1-1) and streptomycin (5 g 1-1) were administered for 3-5 days before peroral bacterial inoculations (−1×10⁸ bacteria in PBS) or PBS alone (sham control) at 4 weeks of age. Fecal bacterial colonization was quantified as colony-forming units per g stool. To define visible colon adenomas, 10% formalin-fixed colons were stained with methylene blue. The adenomas were quantified with a Leica ES2 dissecting scope (by S. W. and C. L. S.) and sized with a Nikon SMZ2 1500 microscope with NIS-Elements AR2.30 software. For histopathology, Swiss-rolled, paraffin-embedded, sectioned (5 μm) and stained colons were stained with H&E. All mouse protocols were approved by the Johns Hopkins University Animal Care and Use Committee in accordance with the Association for Assessment and Accreditation of Laboratory Animal Care International.

### Histopathology.

Inflammation was scored on a 0-4 scale (0, normal mucosa; 1, minimal inflammation (occasional scattered granulocytes and leukocytes); 2, mild inflammation (scattered granulocytes with occasional mild infiltrates); 3, moderate inflammation (scattered granulocytes with patchy moderate infiltrates); and 4, severe inflammation (multiple extensive areas with abundant granulocytes and marked infiltrates)). Colonic proliferation was scored on a 0-3 scale (0, normal mucosa; 1, mild proliferation (patchy distribution of mildly deepened crypts and slightly thicker mucosa); 2, moderate proliferation (regionally diffuse epithelial crowding, deep crypts and thickened mucosa); and 3, severe proliferation (extensive diffuse distribution of marked epithelial crowding, thickened mucosa and markedly elongated, branched crypts)). Gastrointestinal intraepithelial neoplasia (GIN) foci was quantified on one 5-μm section of Swiss-rolled colon.

### Flow Cytometric Analyses.

colons (3-5 mice per group) were processed to obtain mucosal intraepithelial and lamina propria lymphocytes as previously described (Harris, T. J. et al J. Immunol. 179, 4313-4317 (2007)). Mononuclear cells collected by Percoll gradient separation were stimulated with phorbol 12-myristate 13-acetate (PMA) (30 nM), ionomycin (1 μM) and Golgiplug (BD Biosciences) and then stained for cell surface markers and intracellular cytokines. A FACSCalibur (BD Biosciences) was used for flow cytometry and data was analyzed with FlowJo software (Tree Star Inc.). Flow cytometry was also used to analyze mechanically dissociated splenic lymphocytes isolated by density gradient and stimulated with PMA, ionomycin and Golgiplug. Antibodies to the following proteins were used: IFN-γ (clone XMG1.2), IL-17A (clone eBiol7B7), CD4 (clone RM4.5), CD8a (clone 53-6.7), F4/80 (clone BM8), CD11c (clone N418), TCRβ (clone H57-597), TCRγ (clone eBioGL3) (eBiosciences) and IL-4 (clone 11B11), CD3e (clone 145-2C11), CD11b (clone M1/70), NK1.1 (clone PK136), CD16/CD32 (clone CD16/CD32) (BD Biosciences).

### Depletion of T Lymphocytes.

CD3⁺CD4⁺ and CD4⁻CD8⁻TCRαβ-γδ⁺ T lymphocytes were depleted using the GK1.5 antibody ascites (75 μl per dose) and the TCRβ-γδ+ depleting antibody (Clone UC7-13D5; 500 μg per dose), respectively, with rat or hamster IgG isotype antibody (0.5-1 g per dose; Sigma) as a control given intraperitoneally the day before, 1-2 days after then weekly after bacterial inoculations. Spleen and mucosal CD4⁺ or TCRαβ-γδ⁺ depletion by flow cytometry was verified at 1 week and/or 4 weeks of age.

### Cytokine Blockade Protocols.

Monoclonal IL-17A-blocking antibody (clone 50104) and monoclonal IL-23R-blocking antibody (clone 258010) or isotype control antibodies (rat IgG2b, clone 141925; and IgG2a, clone 54447) (R&D Systems) were administered intraperitoneally (500 μg) as described above. Monoclonal IFN-γ-blocking antibody (clone XMG1.2, eBioscience) or rat IgG control antibody were administered using similar methods.

### Real-Time PCR.

We extracted total RNA using the RNAeasy Kit (Invitrogen) and synthesized cDNA. All primers were from Applied Biosystems. We calculated relative gene expression by the ΔΔCT method.

### Detection of Phosphorylated Stat Proteins.

Flash-frozen colonic tissue was processed using phosphatase and protease inhibitors (Roche) to obtain nuclear protein extracts. Western blotting was performed with antibodies specific for pStat1, pStat3, pStat5 (Cell Signaling), pStat4 (Zymed), pStat2 and pStat6 (Abcam) and detected pStat bands with horseradish peroxidase-conjugated goat secondary antibody to rabbit IgG (Jackson Immune Research) with Supersignal West Pico Chemiluminescent Substrate (Pierce). Antibody specificity was verified with cytokine-stimulated cell lines expressing individual pStat proteins. pStat3 immunohistochemistry was performed by antigen retrieval (boiling 0.01 M citrate buffer and 0.025% trypsin) on deparaffinized tissues treated with hydrogen peroxide (0.3%) and 2% goat serum. pStat3 staining was detected with biotinylated goat secondary antibody to rabbit IgG (Southern Biotech), Avidin Biotin Complex (Vector Laboratories) and 3.3′-diaminobenzidine developer, counterstained with hematoxylin.

### Statistical Analyses.

In general, data is presented as box-and-whisker plots, where the line represents the median; the box, the interquartile range; the whiskers, the tenth and ninetieth percentiles; and the dots, individual data points beyond the tenth and ninetieth percentiles. To compare nonparametric distributions across experimental conditions, we used the Mann-Whitney U test. For analysis of graded associations between histology scores and tumor numbers (FIG. 1D), we used a parametric approach: the boxes represent means, the bars represent s.e.m.; the line was derived from a linear regression analysis; and r represents a Pearson correlation coefficient. We considered P values≦0.05 to indicate statistical significance.

### Other Embodiments

From the foregoing description, it will be apparent that variations and modifications may be made to the invention described herein to adopt it to various usages and conditions. Such embodiments are also within the scope of the following claims.

The recitation of a listing of elements in any definition of a variable herein includes definitions of that variable as any single element or combination (or subcombination) of listed elements. The recitation of an embodiment herein includes that embodiment as any single embodiment or in combination with any other embodiments or portions thereof.

All patents and publications mentioned in this specification are herein incorporated by reference to the same extent as if each independent patent and publication was specifically and individually indicated to be incorporated by reference.

1. A method of diagnosing a subject as having, or having a propensity to develop inflammatory bowel disease or colon carcinogenesis, the method comprising detecting an enterotoxigenic B. fragilis (ETBF) nucleic acid molecule in a biological sample from a subject, wherein the presence of the ETBF nucleic acid molecule indicates that the subject has or has a propensity to develop inflammatory bowel disease or colon carcinogenesis.

2. The method of claim 1, wherein the nucleic acid molecule is bft-1, bft-2, bft-3, or a nucleic acid molecule that encodes a related isoform.

3. The method of claim 1, wherein the biological sample is a stool sample or blood sample.

4. The method of claim 1, wherein the ETBF nucleic acid molecule is detected by PCR, qPCR, Northern blot, or probe hybridization.

5. The method of claim 1, wherein the method detects an increase in the level of expression of the ETBF nucleic acid molecule relative to a reference.

6. A method of diagnosing a subject as having, or having a propensity to develop, a inflammatory bowel disease or colon carcinogenesis, the method comprising detecting an ETBF polypeptide in a subject sample, wherein the presence of ETBF polypeptide in the sample indicates that the subject has or has a propensity to develop inflammatory bowel disease or colon carcinogenesis.

7. The method of claim 6, wherein the ETBF polypeptide is BFT-1, BFT-2, BFT-3, or a related isoform.

8. The method of claim 5, wherein the method detects an increased level of the ETBF polypeptide relative to a reference.

9-10. (canceled)

11. A method of diagnosing a subject as having, or having a propensity to develop, a inflammatory bowel disease or colon carcinogenesis, the method comprising detecting an antibody against an ETBF polypeptide in a subject sample, wherein the presence of the antibody in the sample indicates that the subject has or has a propensity to develop inflammatory bowel disease or colon carcinogenesis.

12-13. (canceled)

14. The method of claim 12, wherein the antibody specifically binds BFT-1, BFT-2, BFT-3 or a related isoform.

15. A method of monitoring a subject diagnosed as having inflammatory bowel disease or colon carcinogenesis, the method comprising determining the level of an ETBF nucleic acid molecule or polypeptide in a subject sample, wherein an alteration in the level of expression relative to the level of expression in a reference indicates the severity of inflammatory bowel disease or colon carcinogenesis in the subject.

16. The method of claim 15, wherein the nucleic acid molecule is bft-1, bft-2, bft-3, or a nucleic acid molecule that encodes a related isoform, and the polypeptide is BFT-1, BFT-2, BFT-3, or a related isoform.

17. The method of claim 15, wherein the subject is being treated for inflammatory bowel disease or colon carcinogenesis.

18-22. (canceled)

23. The method of claim 1, wherein the method is used to diagnose a subject as having ETBF-induced colitis, inflammatory bowel disease, colonic hyperplasia or tumor formation.

24-25. (canceled)

26. The method of claim 1, wherein the method further comprises characterizing inflammation, hyperplasia and/or gastrointestinal intraepithelial neoplasia (GIN) foci in said subject.

27. The method of claim 1, wherein the method further comprises characterizing phosphorylated Stat3 (pStat3) in intestinal mucosa of said subject.

28. An ETBF antibody that specifically binds to an ETBF protein or fragment thereof.

29. (canceled)

30. A method for treating or preventing inflammatory bowel disease or colon carcinogenesis, the method comprising administering to a subject an effective amount of an immunogenic composition comprising a nucleic acid molecule encoding a ETBF protein or fragment thereof, or
A method for treating or preventing inflammatory bowel disease or colon carcinogenesis, the method comprising administering to a subject an effective amount of an immunogenic composition comprising an ETBF protein or fragment thereof, or
A method for treating or preventing inflammatory bowel disease or colon carcinogenesis, the method comprising administering to a subject an effective amount of a killed or attenuated ETBF cell, or
A method for producing an immune response against ETBF in a subject, the method comprising administering to the subject an effective amount of an immunogenic composition comprising an ETBF polypeptide, ETBF nucleic acid molecule, and/or killed or attenuated ETBF cell, thereby generating an immune response in said subject

31. (canceled)

32. The method of claim 30, wherein the ETBF protein is BFT-1, BFT-2, BFT-3, or a related isoform.

33-39. (canceled)

40. A method for treating or preventing inflammatory bowel disease or colon carcinogenesis in a subject, the method comprising administering to the subject an effective amount of a STAT3 inhibitor, thereby treating or preventing inflammatory bowel disease or colon carcinogenesis, or
A method for treating or preventing inflammatory bowel disease or colon carcinogenesis in a subject, the method comprising administering to the subject an effective amount of an agent that reduces IL-17 biological activity, thereby treating or preventing inflammatory bowel disease or colon carcinogenesis, or
A method for treating or preventing inflammatory bowel disease or colon carcinogenesis in a subject, the method comprising administering to the subject an effective amount of an agent that reduces IL-23 binding to an IL-23 receptor, thereby treating or preventing inflammatory bowel disease or colon carcinogenesis, or
A method for treating or preventing inflammatory bowel disease or colon carcinogenesis in a subject, the method comprising administering to the subject an effective amount of an agent that reduces the proliferation or survival of ETBF in a subject.

41-48. (canceled)

49. A method of identifying a compound that treats or prevents inflammatory bowel disease or colon cancer, the method comprising contacting a cell that expresses an ETBF nucleic acid molecule or polypeptide with a candidate compound, and detecting a reduction in the level of expression of the nucleic acid molecule or polypeptide in the cell relative to the level of expression in a control cell, wherein a reduction in expression of the ETBF nucleic acid molecule or polypeptide identifies the candidate compound as a compound that treats or prevents inflammatory bowel disease or colon cancer, or
A method of identifying a compound that treats or prevents inflammatory bowel disease or colon cancer, the method comprising contacting a cell that expresses an ETBF polypeptide with a candidate compound, and detecting a reduction in the biological activity of the ETBF polypeptide in the cell relative to the level in a control cell, wherein a reduction in ETBF biological activity identifies the candidate compound as a compound that treats or prevents inflammatory bowel disease or colon cancer, or
A method of identifying a compound that treats or prevents inflammatory bowel disease or colon cancer, the method comprising contacting a colon-derived cell that expresses a STAT3, NFκB, MAPK or Wnt polypeptide with a candidate compound, and comparing the level of STAT3, NFκB, MAPK or Wnt expression or biological activity in the cell with the level of STAT3, NFκB, MAPK or Wnt expression or biological activity in a control cell, wherein a decrease in the expression or activity of the STAT3, NFκB, MAPK or Wnt polypeptide identifies the candidate compound as a compound that treats or prevents inflammatory bowel disease or colon cancer.

50-55. (canceled)

56. A diagnostic kit for the diagnosis of inflammatory bowel disease or colon carcinogenesis in a subject comprising one or more primers for amplifying an ETBF nucleic acid molecule, or fragment thereof, and written instructions for use of the kit in a method of claim 1, or
A diagnostic kit for the diagnosis of inflammatory bowel disease or colon carcinogenesis in a subject comprising an ETBF polypeptide bound to a substrate and directions for the use of said kit for the detection of an antibody that specifically binds to said ETBF polypeptide in a subject sample, or
A kit for treating or preventing inflammatory bowel disease or colon carcinogenesis in a subject comprising an ETBF polypeptide, ETBF nucleic acid molecule, or ETBF cell formulated as an immunogenic composition, and written instructions for use of the kit to induce an immune response in a subject.

57-63. (canceled)
